(19) United States
(12) Patent Application Publication
(10) Pub. No.: US 2026/0279509 A1
(43) Pub. Date: Sep. 17, 2026

(54) MOLECULAR DESIGN WITH QUANTUM-COMPUTING-BASED MACHINE LEARNING AND OPTIMIZATION

(71) Applicant: CORNELL UNIVERSITY, Ithaca, NY (US)

(72) Inventors: Fengqi YOU, Ithaca, NY (US); Akshay AJAGEKAR, Ithaca, NY (US)

(21) Appl. No.: 19/162,620

(22) PCT Filed: Mar. 7, 2024
(86) PCT No.: PCT/US2024/018975

§ 371 Date: Sep. 5, 2025

Related U.S. Application Data
(60) Provisional application No. 63/450,421, filed on Mar. 7, 2023.

(51) Int. Cl. G16C 20/50; G16C 20/70

(52) U.S. Cl. CPC G16C 20/50; G16C 20/70

(57) ABSTRACT

A method for molecular design includes constructing a structural cost function having first minima that identify molecular species satisfying structural constraints. The method includes generating exploratory molecular species by minimizing the structural cost function to obtain arguments of one of the first minima; creating, based on the arguments, an exploratory feature vector; and evaluating an objective function with the exploratory feature vector, a target range of a molecular property, and parameters of a machine-learning model to obtain an objective-function value. The method includes generating a candidate molecular species by performing regression with the objective-function values to update a surrogate model that approximates the objective function; constructing an error cost function having second minima that identify molecular species that minimize the surrogate model while satisfying the structural constraints; and minimizing, with a quantum computer, the error cost function to identify one of the second minima.

### RELATED APPLICATIONS

This application claims priority to U.S. Provisional Patent Application No. 63/450,421, filed Mar. 7, 2023 and titled “Molecular Design with Automated Quantum Computing-Based Deep Learning and Optimization,” which is incorporated herein by reference in its entirety.

### BACKGROUND

Quantum computing has the potential to achieve significant technological feats in various domains, including the design of novel molecules.

### SUMMARY

A method for molecular design includes constructing a structural cost function having a plurality of first minima, each of the plurality of first minima identifying a molecular species that satisfies one or more molecular-structure constraints. The method also includes generating each of a plurality of exploratory molecular species by minimizing the structural cost function to obtain one or more arguments of one of the plurality of first minima; creating, based on the one or more arguments of said one of the plurality of first minima, an exploratory feature vector that identifies said each of the plurality of exploratory molecular species within a feature space; and evaluating an objective function with (i) the exploratory feature vector, (ii) a target range of a molecular property, and (iii) model parameters of a trained machine-learning model to obtain a respective one of a plurality of objective-function values. The method also includes generating a candidate molecular species by performing regression with a randomly selected subset of the plurality of objective-function values to update a surrogate model that approximates the objective function; constructing an error cost function by combining the surrogate model, as updated by the regression, and the structural cost function such that each of a plurality of second minima of the error cost function identifies a molecular species that minimizes the surrogate model while satisfying the one or more molecular-structure constraints; and minimizing, with a quantum computer, the error cost function to obtain one or more arguments of one of the plurality of second minima. The method also includes outputting the one or more arguments of said one of the plurality of second minima. The trained machine-learning model was trained with a plurality of training samples, each training sample of the plurality of training samples comprising (i) a training feature vector that identifies a training molecular species of said each training sample within the feature space and (ii) a training target vector that specifies a predefined range of values of the molecular property, the training molecular species having a value of the molecular property lying within the predefined range.

### BRIEF DESCRIPTION OF THE FIGURES

FIG. 1A illustrates example fixed-length neural fingerprints generated by a GraphConv network with fixed weights, in embodiments.

FIG. 1B illustrates training of an energy-based model by drawing samples from a quantum annealer, in embodiments.

FIG. 1C illustrates construction of a surrogate model to estimate the free energy of a molecule-property pair with the trained energy-based model, in embodiments.

FIG. 2A is a table summarizing molecule generation efficacy.

FIG. 2B illustrates two-dimensional (2D) embeddings obtained with t-distributed stochastic neighbor embedding (t-SNE) for the molecules in the training set as well as the generated molecules colored according to the quantitative estimation of drug-likeness (QED) property values, in embodiments.

FIG. 3A is a KDE plot illustrating the density of partition coefficient (Log P) and QED for molecules in the training set.

FIG. 3B provides KDE plots indicate the Log P and QED for generated molecules.

FIG. 3C shows violin plots for target conditions of the distribution of synthetic accessibility scores (SAS) of QED for the generated molecules.

FIG. 3D shows violin plots for target conditions of the distribution of SAS of Log P for the generated molecules.

FIG. 3E illustrates examples of generated molecular structures conditioned upon restrictions on molecular properties of QED, in embodiments.

FIG. 3F illustrates examples of generated molecular structures conditioned upon restrictions on molecular properties of partition coefficient (Log P), in embodiments.

FIGS. 4A-E illustrate distributions of the molecular properties of molecules generated with various molecular design frameworks, including the QC-based technique of the present embodiments, semi-supervised variational autoencoder (SSVAE), and conditional variational autoencoder (CVAE), where the molecules are generated with these frameworks for different property targets for drug-likeness (QED), in embodiments.

FIGS. 4F-J illustrate distributions of the molecular properties of molecules generated with various molecular design frameworks, including the QC-based technique of the present embodiments, semi-supervised variational autoencoder (SSVAE), and conditional variational autoencoder (CVAE), where the molecules are generated with these frameworks for different property targets for partition coefficient (Log P), in embodiments.

FIG. 5A illustrates learning curves for the conditional energy-based model trained with QC-assisted generative training and CD-learning, in embodiments.

FIG. 5B illustrates an optimization trajectory of a molecular design problem with QC-assisted optimization and constrained Bayesian optimization, in embodiments.

FIG. 5C illustrates a comparison of computational times required to solve the QUBO problems of varying sizes occurred during molecular generation for different atomic compositions and property targets with both simulated annealing implemented on a classical computer and quantum annealing, in embodiments.

FIG. 6A illustrates a principal component plot of the first three components of latent variable representations for 1,000 randomly selected molecules organized by molecular properties of quantitative drug-likeness (QED), in embodiments.

FIG. 6B illustrates a principal component plot with the first three components of latent variable representations for 1,000 molecules organized by molecular properties of synthetic accessibility scores (SAS), in embodiments.

FIG. 6C illustrates a principal component plot with the first three components of latent variable representations for 1,000 molecules organized by molecular properties of partition coefficient (Log P), in embodiments.

FIG. 7 illustrates example distribution of the Tanimoto similarities obtained for different molecular pairs with molecular access system (MACCS) keys, extended-connectivity fingerprints (ECFPs), and neural fingerprints, in embodiments.

FIG. 8 illustrates a conditional restricted Boltzmann machine.

FIG. 9 is a flowchart of a method for exploratory molecular design, in embodiments.

FIG. 10 is a flowchart of a method for generative molecular design, in embodiments.

FIG. 11 is a functional diagram of a system for molecular design, in embodiments.

### DETAILED DESCRIPTION

### Introduction

The development of novel compounds and materials has played a critical role in the advancements of various scientific fields. Technological and societal progress can be further fueled by the discovery of novel molecules for applications ranging from drug design for treating diseases to efficient energy storage devices for combating climate issues [1]. The potential for the synthesis of novel molecular compounds remains vast; for instance, the number of molecules with pharmacological properties that could be synthesized is estimated to be as high as 10⁶⁰ [2]. Computer-aided molecular design can facilitate the generation of molecular candidates with desired chemical properties through various means [2]. Simulations of chemical systems could estimate molecular properties through quantum chemistry calculations. However, these techniques are unable to handle large-scale systems through first-principle calculations and require the use of approximations adopted at the expense of accuracy, which may inhibit efficient exploration of the chemical space [4]. Computational optimization techniques have also demonstrated some success in the generation of useful molecules [5]. Additionally, machine learning, specifically deep learning, has accelerated progress in molecular design through learning patterns in molecular datasets and offers a promising route towards the development of novel compounds [6]. The complexities stemming from the nonlinearity of molecular design problems at larger scales lead to the intractability of optimization techniques [7], while the machine learning techniques may not be data-efficient and yield inaccurate predictions despite consuming prohibitively large computational power to process huge amounts of molecular data. It is imperative to exploit efficient computational techniques for a guided exploration of the chemical space to generate insights into the synthesis of novel molecules.

Quantum computing (QC) holds tremendous potential to achieve significant technological feats in various domains, including the design of novel molecules for specific purposes [8]. Owing to their ability of exploiting quantum mechanical phenomena for performing computation, QC techniques have demonstrated remarkable improvements for several applications. The promise of performance enhancement offered by QC has also attracted considerable attention from the research community for the development of QC-based methods in fields like computational chemistry, optimization, and machine learning [9]. Quantum computers offer a fundamentally different approach for performing quantum chemistry simulations that have helped overcome the practical challenges of simulating chemical systems on classical computers [10]. Quantum algorithms have also facilitated the development of quantum-enhanced optimization and machine learning techniques tailored for specific problem types and learning tasks [11,12]. Despite their advantages, QC techniques implemented on current quantum devices exhibit limitations in terms of performance and scalability due to the presence of hardware noise and a limited number of quantum bits or qubits [13]. Therefore, it is important to develop models and methods that harness the complementary strengths of high-performance quantum and classical computation by overcoming their individual limitations in order to effectively and efficiently navigate through the complex chemical space for molecular design. Although quantum-enhanced machine learning and optimization can be employed for molecular property prediction and inverse design, several research challenges remain. Developing prediction models and design methods that are compatible with near-term quantum devices with noisy qubits is the first challenge. There have been attempts on hybrid quantum-classical optimization techniques for determining the structural configuration of molecules [14,15], but these approaches do not scale for larger molecules on today's quantum computers. As a result, scalable QC approaches for molecular design that can handle problems across varying scales is another important research challenge. An additional one lies in exploiting the noisy nature of near-term intermediate-scale quantum (NISQ) devices, which typically have between fifty and a few hundred qubits without fault-tolerant capabilities for learning and inverting structure-property relationships without compromising performance.

The present embodiments include hybrid quantum-classical computational framework for molecular design that utilizes QC-based learning and optimization strategies to efficiently navigate the chemical space for targeted molecular generation. We, the inventors, constructed an energy-based deep learning model that can be trained with QC-assisted generative training to extract latent representations from molecular graphs for property prediction. A QC-assisted optimization technique is further presented that efficiently traverses through the chemical space to identify molecules with desired properties by inverting the structure-activity relationship captured with the energy-based model. These are particularly advantageous when exploring the chemical space for candidate molecules by not relying solely on the available molecular datasets. A thorough analysis that includes benchmarking against the baseline models and investigating the efficacy of the molecular generation pipeline is also conducted. The deep learning model learns the conditional distribution of molecular properties depending on their structural information allowing us to efficiently predict properties for a given molecule. Consequently, it learns the relationship between molecular composition and structure and its physiochemical properties, which is exploited by the optimization technique of the present embodiments to generate molecules exhibiting target properties. Upon benchmarking the obtained computational results with existing deep learning-based approaches, the molecular design framework of the present embodiments not only demonstrates competitive predictive performance for physiochemical properties but also efficiently generates molecules for predefined property requirements. The computational experiments conducted to validate the generated molecules and their properties obtained with various molecular design approaches also reveal the data efficiency and generalization capabilities of the QC-based molecular design approach through the exploration of sparsely populated regions of the chemical space.

### Related Work

Reliable techniques for molecular property prediction and efficient search strategies are the building blocks for computer-aided molecular design [16]. Prediction models that can estimate the properties of given molecules can assist the virtual screening process in isolating candidate molecules with desired properties [17]. Computational screening of molecules is dependent on the quality of virtual chemical libraries manually constructed from chemical databases or through combinatorial approaches [19,20], and may induce uncertainty in the exploration of the appropriate chemical space. In addition to using first-principle simulations for predicting molecular properties [21], machine learning techniques have also led to the development of advanced prediction models that estimate properties with competitive precision as that of traditional techniques [22]. Such data-driven methods circumvent the need for computationally expensive quantum chemical methods and have been more commonly embraced to improve the performance of predictive models [24]. Characterization of molecular structure-property relationships through interpretation of machine learning models can be further used to guide the design of novel molecules and is referred to as inverse molecular design [25]. Inverse design can be performed by navigating the chemical space of molecules with target functionality through optimization, search, or sampling techniques [26]. Several optimization techniques, including both heuristic and deterministic algorithms, can be applied to inverse molecular design cast as an optimization problem. Evolutionary techniques like genetic algorithms and discrete combinatorial optimization approaches like mixed-integer programming have demonstrated their utility for the design of various molecules. However, genetic algorithms require manual adjustment of heuristic rules for different optimization problems and do not guarantee optimality, while combinatorial optimization approaches may exhibit difficulty in solving large-scale nonlinear optimization problems [29]. These computational challenges can be tackled by deep learning methods that utilize sophisticated neural network architectures for constructing generative models for molecular design. Various deep generative models allow for adopting different molecular representations as input and can be trained to learn the distribution of the molecular dataset [6], which is followed by random sampling of molecules for further screening with property estimation models. Recurrent neural networks [30,31] and variational autoencoders [32,33] that use SMILES identifier as input, and graph convolutional neural networks (GraphConv) that operate on molecular structures represented by graphs are some of the commonly used deep learning architectures for molecular design. Deep learning approaches can also be tailored for the conditional generation of molecules that satisfy the target property requirements [35]. In addition to direct sampling from trained generative models, Bayesian optimization and reinforcement learning [36], assisted by neural network architectures for property estimation, can perform a guided search for molecules through additional optimization steps often carried out in the latent space of the neural networks. Recently, QC algorithms have also been proposed for the search of optimal configuration of molecules in protein chains that demonstrate a quantum speedup over straightforward enumeration [37].

Despite their potential, the dependence of deep neural networks on large amounts of diverse training data comprising of molecules with properties spanning the chemical space may cast doubt on their generative abilities in the presence of out-of-domain uncertainty. Exploiting advantages offered by QC for molecular design remains a major research challenge for existing QC-based approaches with near-term quantum devices. Unlike these methods, the QC-based molecular design framework utilizes NISQ devices to learn from limited molecular data and search for molecular candidates with target properties through optimization in a robust manner. Additionally, previous deep learning methods for molecular generation do not always facilitate constrained sampling of molecular candidates [6]. In contrast, our molecular design framework is designed to generate molecules given a certain composition that exhibit target property values within specific ranges.

### Materials

Data: We use compounds from the Zinc database to train and validate the performance of the present embodiments. A subset of Zinc having 12,000 molecules that are commonly used for benchmarking purposes is collected for our computational study [39]. The collected SMILES identifiers of the molecules are converted to graph-structured data by identifying the node features and edge features using the RDKit package [40]. The node features distinguish constituent atoms into nine heavy atom types, while the edge features describe the presence of bonds between atoms and bond types. For each molecular graph in the dataset, we also collected three different properties, namely, the quantitative estimation of drug-likeness (QED) [41], Wildman-Crippen partition coefficient (Log P) [42], and the synthetic accessibility score (SAS) [43]. The Log P value or the water-octanol partition coefficient provides a measure of lipophilicity and serves as one of the molecular properties used to estimate QED. On the other hand, the SAS values describe the ease of synthesis of drug-like molecules. The QED property can vary between zero and one, with a larger value indicating a more drug-like molecule while the SAS ranges from one to ten. Among the molecules in the selected dataset, Log P ranges from −4.58 to 6.66. The averages of QED, SAS, and Log P for the collected samples are 0.728, 3.048, 2.450, and the standard deviations for these are 0.138, 0.834, and 1.433, respectively. Target labels for each molecule and its corresponding property are also generated by discretizing the target space into several bins. Bin ranges for each property are provided in Table S1.

Baseline methods: To evaluate the efficiency of the structure-property relationship captured by the energy-based model, we benchmark their predictive performance against different neural network models that adopt various learning strategies and input types. The latent vector representations of the conditional energy-based models trained with both contrastive divergence (CD) learning and QC-based generative training are used as inputs to a single-layered neural network for predicting the corresponding property targets. CD-learning is an approximate learning approach and has been extensively used for training energy-based models [45], so it is chosen for comparison against the energy-based model trained with quantum generative approach. Additionally, three baseline models, including feedforward neural networks that use extended-connectivity fingerprints (ECFPs) and molecular access system (MACCS) keys molecular representations as input, as well as a GraphConv model [48] that operates directly on molecules cast as graph-structured data to predict their properties directly, are chosen as baselines for property estimation performance.

In addition to revealing the exploration capabilities of the QC-based molecular design approach in the sparsely populated chemical space, it is important to validate the targeted molecular generation. To this end, we compare the present embodiments with two baseline deep learning-based approaches for molecular design, namely, semi-supervised variational autoencoder (SSVAE) and conditional variational autoencoder (CVAE) for benchmarking the targeted molecular generation results. SSVAE is trained with partial annotations and simultaneously performs both property prediction and molecule generation, while the CVAE approach controls multiple molecular properties by imposing them on the latent space. Both autoencoder-based baselines are generative models operating on the SMILES representations of molecules due to their compact line notation obtained with simple vocabulary and grammar rules [50].

### Computational Results

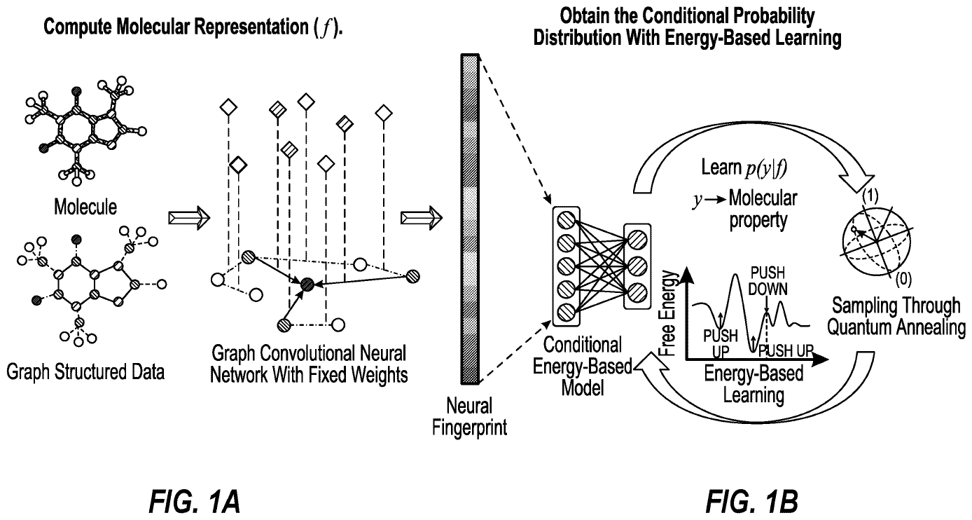

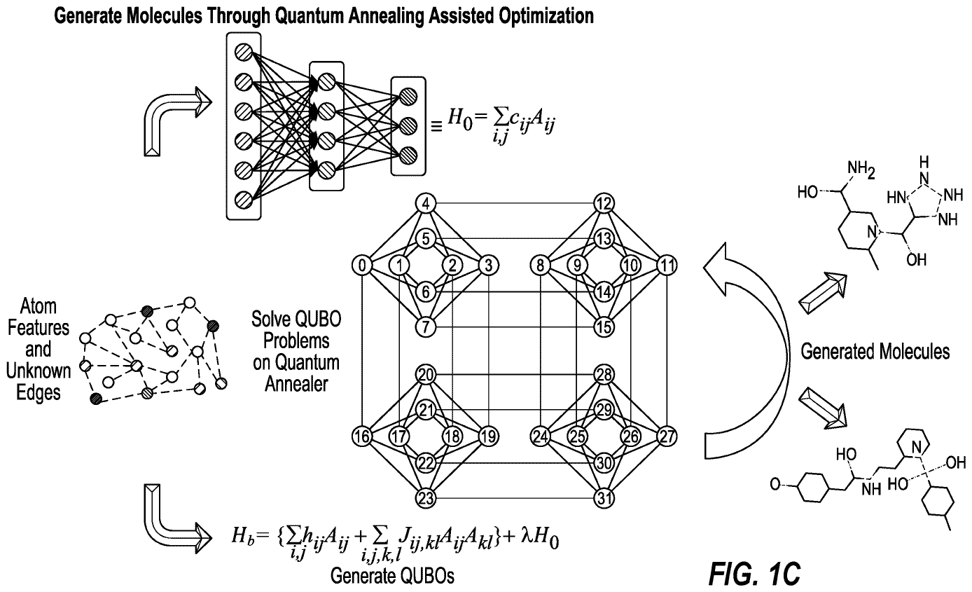

QC-Assisted Molecule Generation Framework: This study utilized quantum annealing based strategies for learning and optimization required for molecular generation. We first construct an energy-based model to learn the distribution of molecular properties conditioned on corresponding representations. A GraphConv network with fixed weights is employed to generate fixed-length neural fingerprints, as illustrated in FIG. 1A. The only input to this model is the structural information of the molecule describing the atom types and their connectivity [34]. The constructed energy-based model uses the generated molecular descriptors f and the molecular properties y as the input data. This energy-based model is trained with a set of molecule-property pairs by drawing samples from a quantum annealer. Upon training, the constructed energy-based model learns the probability distribution p (y| f), as shown in FIG. 1B. The conditional energy-based model also utilizes latent variable representations that can be considered as the compressed chemical space spanned by the molecules and their properties. These latent representations can be further used to perform molecular property estimation tasks. For molecular generation, we employ an iterative optimization procedure that utilizes a quantum annealer to solve formulated quadratic unconstrained binary optimization (QUBO) problems. As illustrated in FIG. 1C, a surrogate model to estimate the free energy of the molecule-property pair with the trained energy-based model is constructed. Governed by the optimization procedure of the present embodiments, the surrogate model is sequentially refined to explore the chemical space for identifying molecules that satisfy the desired property requirements and structural constraints.

TABLE 1
Mean absolute errors for the predicted property targets with different
molecular descriptors and inputs generated with both conventional
and QC-based techniques. The prediction metrics are computed
over a test set comprising 1,000 molecules. Lower values indicate
better predictive performance of the model that uses specified
inputs obtained with the corresponding method.

| Method* | Input (Size) | QED | SAS | LogP |
|---|---|---|---|---|
| Rule-based | ECFP¹ (2048) | 0.17 ± 0.003 | 0.89 ± 0.21 | 1.18 ± 0.08 |
| Rule-based | MACCS² (512) | 0.19 ± 0.008 | 0.76 ± 0.01 | 1.24 ± 0.02 |
| GraphConv | Neural (256) | 0.75 ± 0.02 | 0.88 ± 0.02 | 0.81 ± 0.01 |
| CD-learning | Latent | 0.11 ± 0.01 | 0.82 ± 0.16 | 1.19 ± 0.03 |
| | variables (64) | | | |
| QC-assisted | Latent | 0.10 ± 0.003 | 0.79 ± 0.11 | 1.18 ± 0.01 |
| learning | variables (64) | | | |

¹Extended-connectivity fingerprints (ECFP) describe the presence of particular substructures. ⁴⁶
²MACCS keys indicate the presence or absence of specific chemical groups. ⁴⁷
*Training of the feedforward networks that use the above inputs obtained with the corresponding method was conducted with 10,000 molecules in the training set.

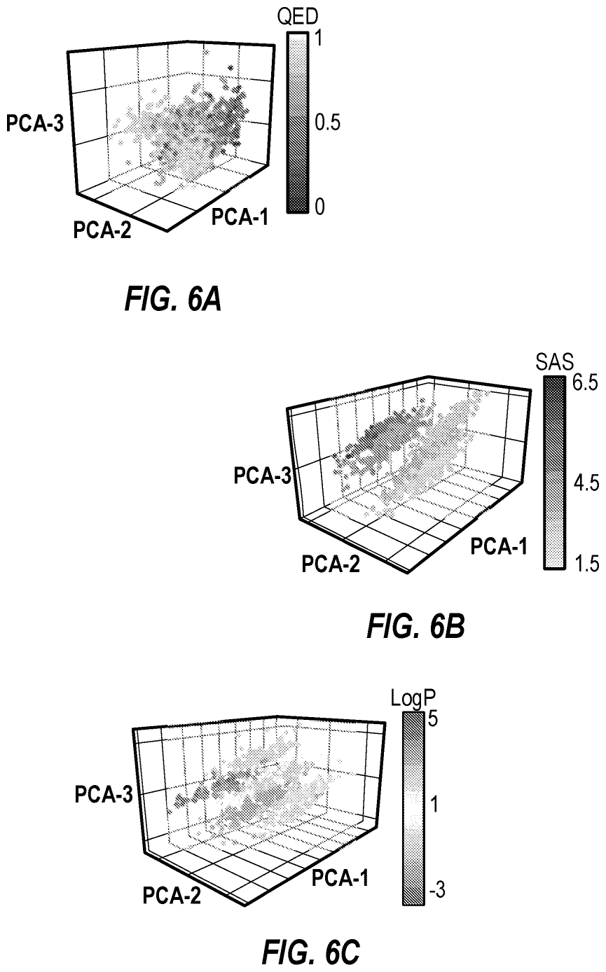

Molecular Property Prediction: Constructing an efficient molecular property prediction model that can provide insights into the structure-property relationships is an important first step towards guiding the generation of molecules with desired properties. The latent representations of the molecules generated by the conditional energy-based model plays an important role in predicting molecular properties. The predictive performance of feedforward models that use various inputs obtained through different methods is presented in Table 1. The average and standard deviation values of the mean absolute error computed over multiple training-evaluation repetitions are reported here. Among the baseline predictive models that use rule-based molecular descriptors as input, the larger ECFP fingerprint tends to perform better for predicting QED and Log P, while the predictive model with MACCS yields much more accurate predictions for the accessibility scores. The baseline model, which uses neural fingerprints generated by the GraphConv model, produces significantly higher errors when predicting the drug-likeness property of the molecule but obtains competitive results for the remaining molecular properties with only 13.6% increase over the lowest error associated with the synthetic accessibility scores. On the other hand, the latent variable representations obtained with the conditional energy-based models trained with both classical and QC-assisted learning techniques achieve an accurate predictive performance for all property targets despite their low dimensionality. The predictive models that use latent representations obtained with QC-assisted generative training not only yield competitive prediction errors as that of other baseline models but also enjoy the least observed error when predicting drug-likeness of the molecules. The obtained computational results demonstrate the effectiveness of the latent representations obtained with the conditional energy-based model trained with QC-assisted learning for molecular property prediction. We also generate the latent representations of 1,000 molecules in the test set with their known target labels using the conditional energy-based model trained through QC-assisted generative training. The mapping of these representations along their three principal components are plotted in FIGS. 6A-6C for various molecular properties.

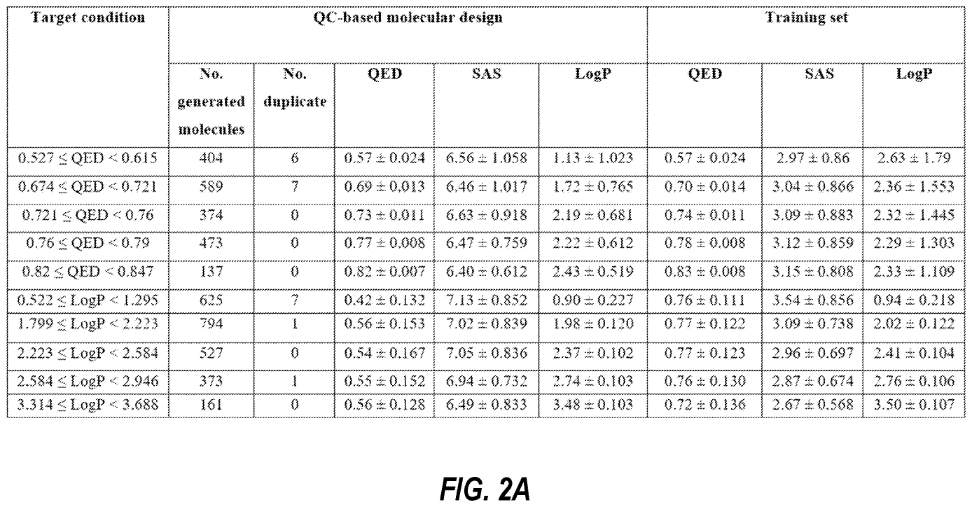

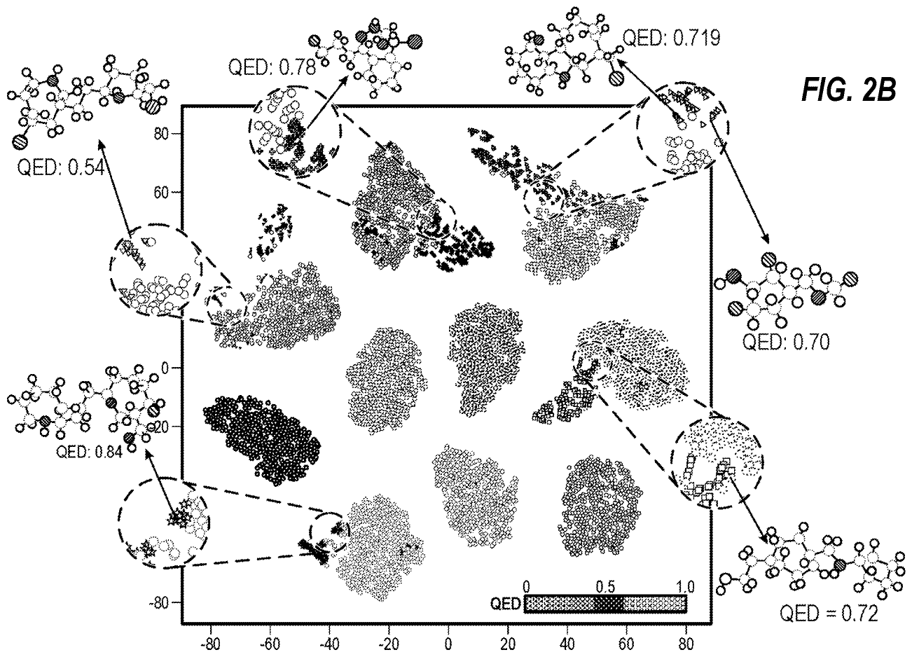

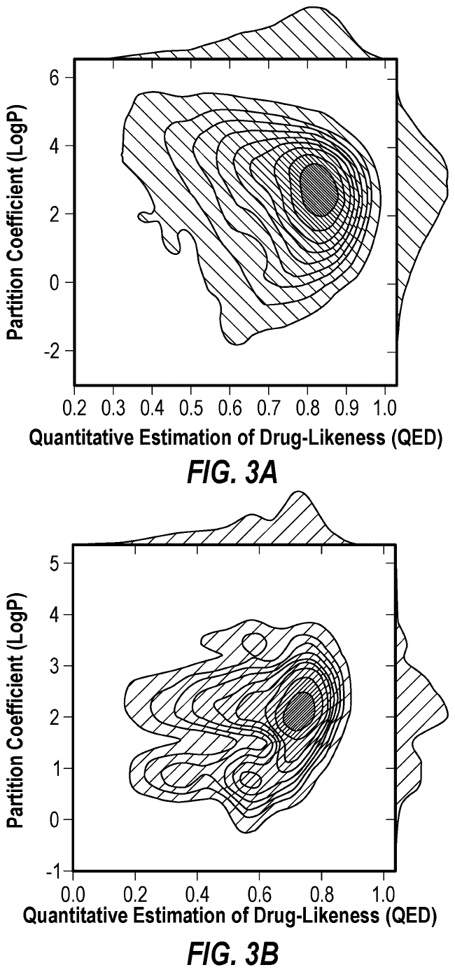

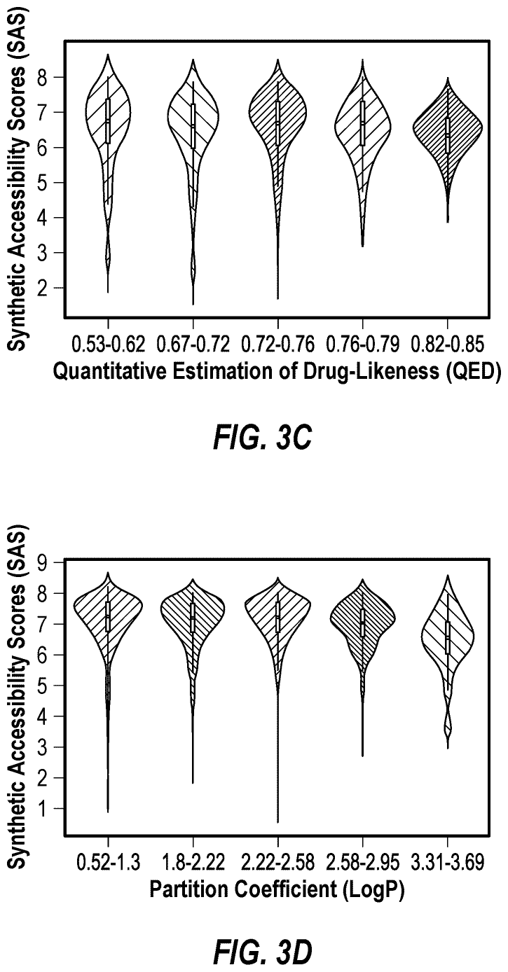

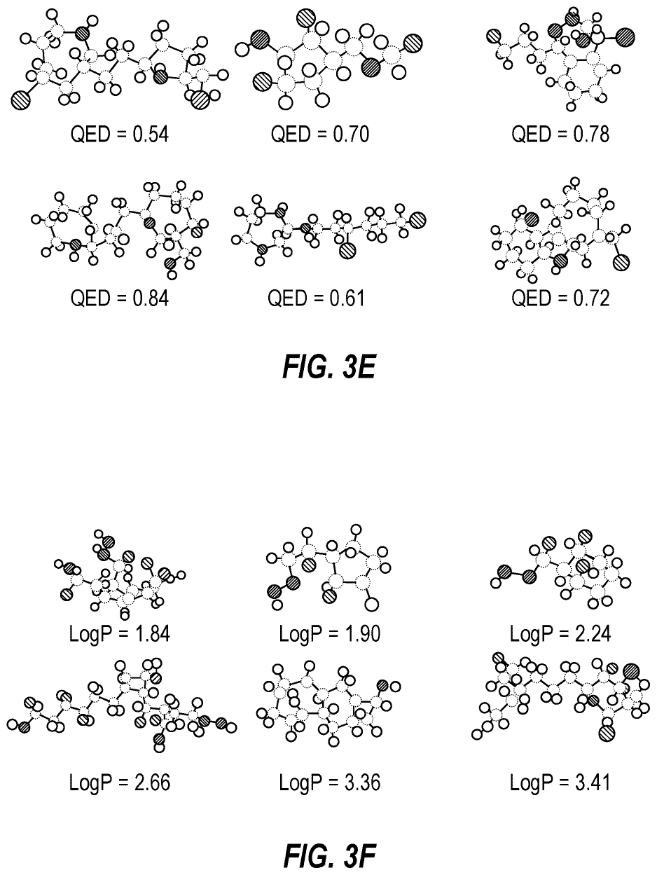

Targeted Molecule Generation: We utilize the trained energy-based model and impose restrictions on the molecular properties for targeted molecular generation with the QC-assisted optimization technique of the present embodiments. Several target conditions are imposed on the drug-likeness of molecules as well as their partition coefficients. FIG. 2A is a table that summarizes the generation statistics for the molecules generated with the QC-assisted optimization technique along with their computed properties. This table also comprises of the identical statistics for the molecules in the training set that satisfy the corresponding target requirements. The number of generated molecules obtained by exploring the chemical space described by each property target range and the atomic identities of the reference molecules in the test set are reported in this table. Among the generated molecules for a given target condition, a few duplicates are also identified that form less than 1.5% of the overall molecules. During the sampling of molecules performed by the QC-assisted approximate optimization technique, the validity of the molecules is verified with the RDKit package [40]. Although invalid molecules that fail to comply with structural constraints are rarely detected owing to the feasible molecular generation capability of the QC-assisted approach, they are automatically discarded to prevent inaccurate estimations computed within this data-driven approach. Furthermore, the average and standard deviation values presented for each target condition are calculated over the observed properties of the generated molecules to ensure the reliability of the molecular generation process subject to conditions. As evident from the computational results in FIG. 2A, the molecules generated for each conditioned property range satisfy the respective imposed restrictions. We also plot the distribution of the SAS scores for all generated molecules and different target properties in FIGS. 3C and 3D to generate insights for their ease of synthesis. From these violin plots, it can be seen that the molecules generated with lower QED and Log P values exhibit wider variability of accessibility scores, despite their relatively higher average SAS scores. A reverse trend is observed for molecules generated for higher utility as drug-like molecules and high Log P values, demonstrating lower variability of SAS scores with low average scores. We also investigate the latent representations for the generated molecules obtained with the trained conditional energy-based model using t-SNE embeddings. Mapping these latent representations in 2D allows us to identify the closeness of molecules with respect to their corresponding properties. FIG. 2B shows the 2D embeddings obtained with t-distributed stochastic neighbor embedding (t-SNE) for the molecules in the training set as well as the generated molecules colored according to the QED property values. We also include a few examples of molecular structures obtained for different property targets in this figure. A distinction between the latent representations for various property ranges can be observed with molecules exhibiting similar QED values adjacent to each other. This illustrates that the constructed energy based-model learns to capture the relationship between the molecules and their properties as molecules with similar properties have close embeddings.

To assess the effects of data used to capture the structure-property relationship on the exploration of the chemical space, the densities of partition coefficients and QED values are visualized with kernel density estimation (KDE) plots along with their marginal distributions for the molecules in the training set and all generated molecules in FIGS. 3A and 3B. The observed concentration of molecules in both training and generated sets is highest in approximately similar ranges of molecular properties. Among the generated molecules, increased density levels are observed for samples with low partition coefficients and high QED values as well as for samples with high partition coefficients and lower QED values. However, this trend is missing for the samples in the training set. This result indicates that, unlike deep generative models that sample molecules from the distribution captured from the training set, the QC-based optimization technique is able to efficiently traverse through the unexplored chemical space that is not previously mapped with the structure-property relationship described by the training set samples. Examples of generated molecular structures sampled for various atomic identities and target properties are also provided in FIGS. 3E and 3F. Although the approximate technique for molecular generation requires extra steps as compared to direct sampling from deep generative models, the QC-assisted technique yields a diverse set of feasible molecules that satisfy structural constraints through efficient guided optimization performed within the target chemical space.

### DISCUSSION

The present embodiments use a machine-learning and optimization framework that exploits QC-based techniques for molecular property estimation and design. We constructed an energy-based model that can be trained with QC-assisted generative training to produce an expressive latent space representation for accurate molecular property prediction. This model is further utilized to guide the chemical space exploration associated with target properties through the QC-based optimization technique of the present embodiments. The resulting latent representations generated by the energy-based model yield high prediction accuracies for different molecular properties as compared to other deep learning models trained with conventional classical learning methods. The viability of the QC-based optimization technique was also demonstrated by generating 4,457 molecules for several target property requirements.

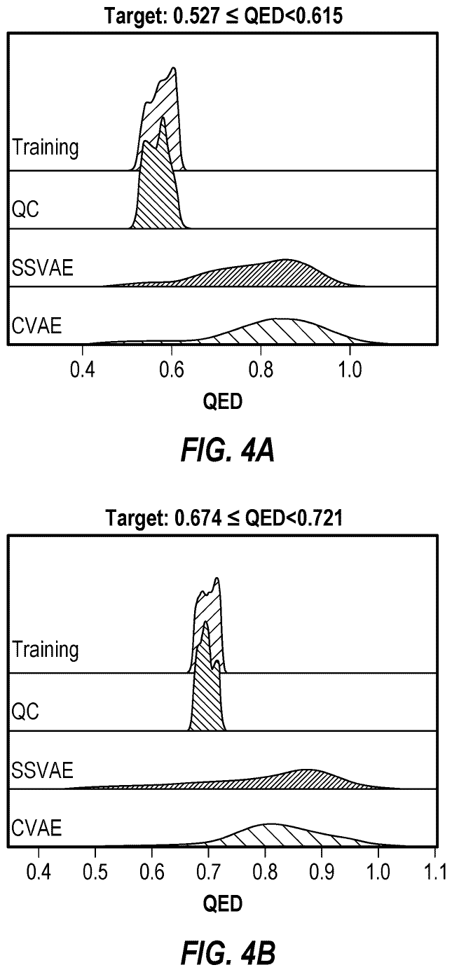

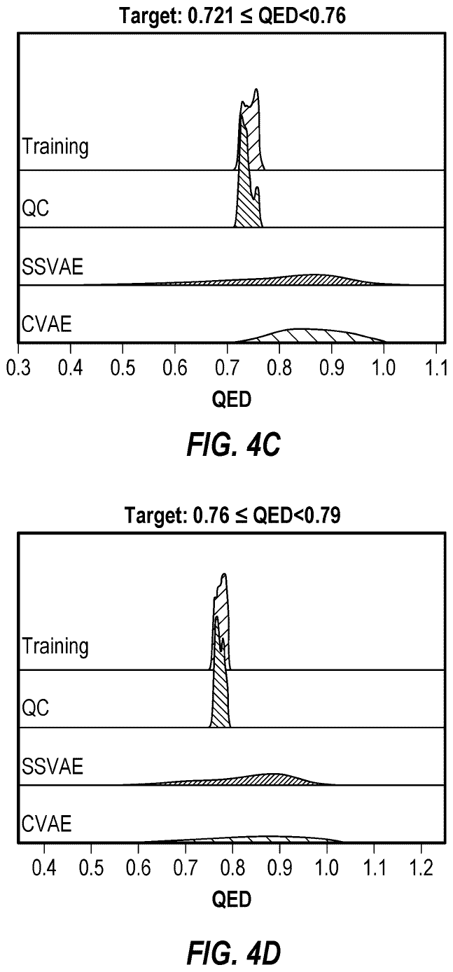

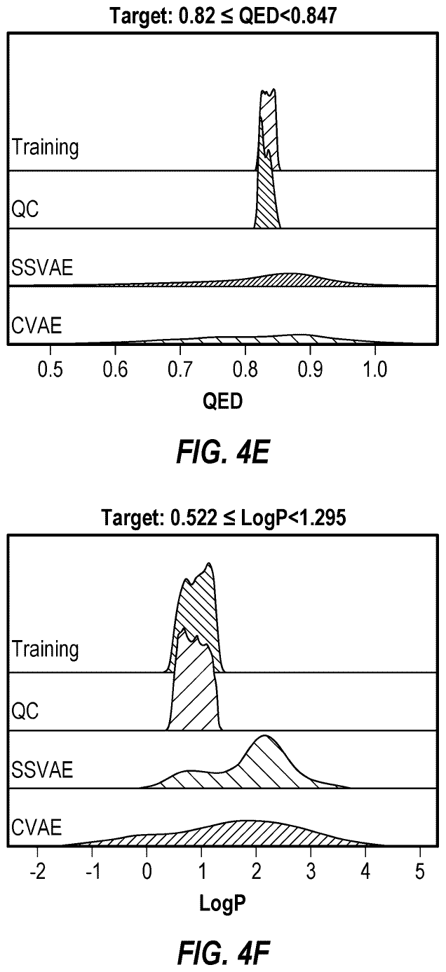

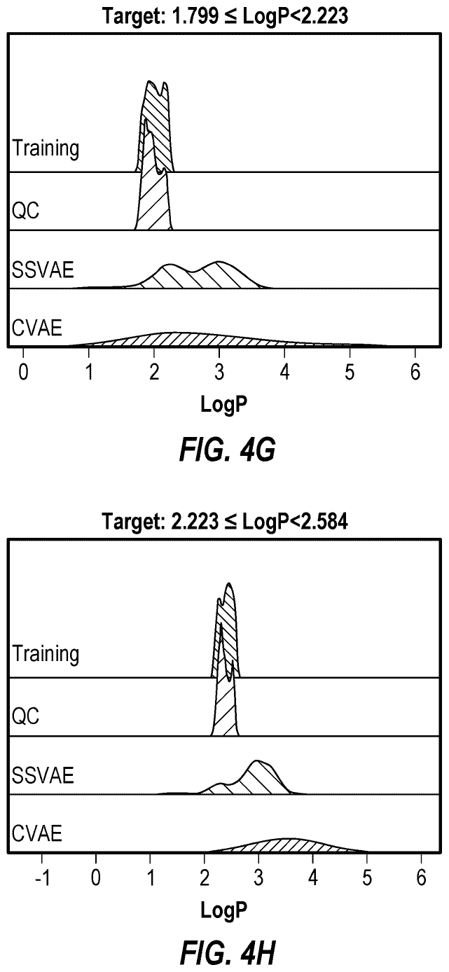

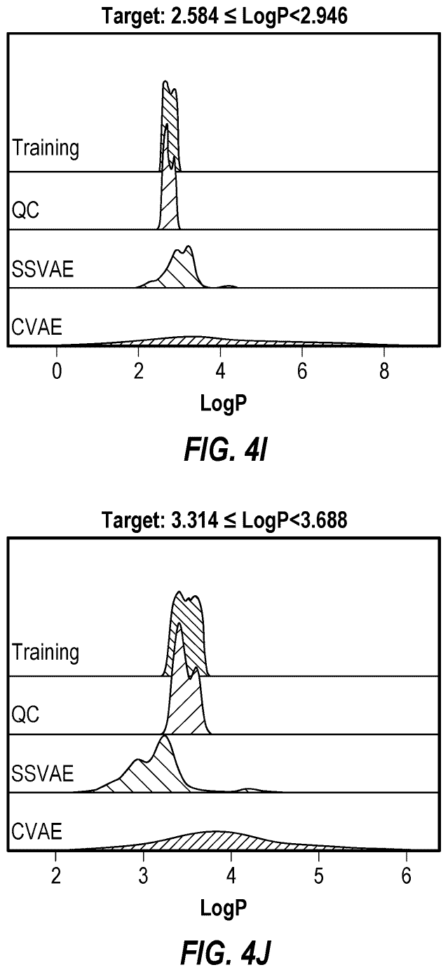

Validation: The validity of the generated molecules obtained with different molecular design frameworks were computed along with their properties, followed by plotting the distributions of the properties for corresponding property targets in FIGS. 4A-J. Distributions for the training data are also plotted to validate the generalization capabilities of the molecular design techniques. As evident from these density plots, the SSVAE and CVAE architectures do not guarantee constrained sampling of molecular candidates. As evident from the efficacy metrics in Table S3, a deviation as high as 40.3% from the mean target property requirements can be observed with the VAE-based deep learning methods for molecular generation. On the other hand, the QC-based approach is able to generate molecules that exhibit target properties efficiently with observed zero violations of the required target property constraints. Additionally, the molecules generated with the molecular design technique follow trends similar to that of the training data, which evidently demonstrates the generalization and data efficiency of the energy-based model trained with QC-assisted learning. The efficacy of the presented QC-based techniques implemented on noisy near-term quantum devices like quantum annealers has further illustrated the promise of QC for the design of novel molecules in the NISQ as well as the fault-tolerant era.

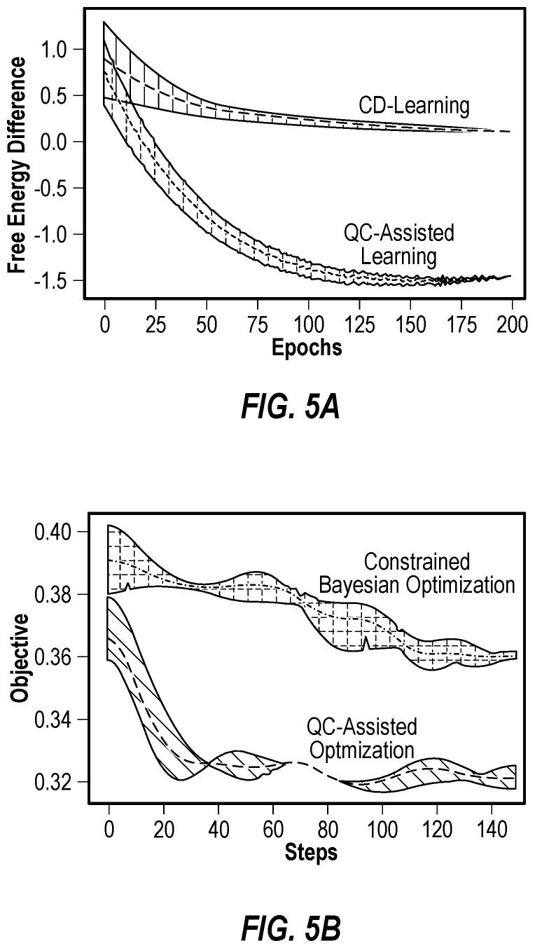

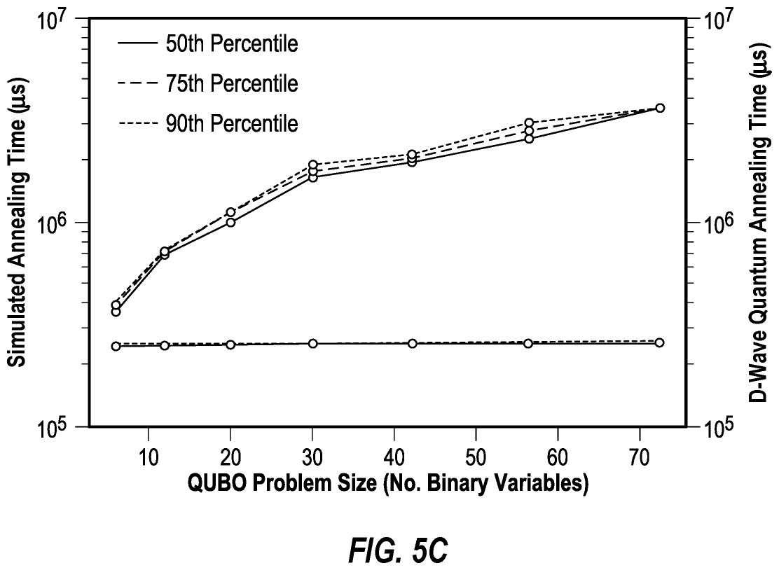

The computational results presented in the above subsections have demonstrated the applicability of QC-assisted techniques for molecular property estimation and design. Although the goal of this work is to develop a molecular design framework capable of exploiting QC techniques for efficient molecular property estimation and generation, it is essential to justify the use of QC for learning and optimization tasks. To this end, we compare the performance of the QC-based techniques with that of their classical counterparts. Computational experiments described herein use quantum annealers for training the conditional energy-based model as well as for sampling molecules. As seen in Table 1, the predictive performance of the model that uses latent representations generated through QC-assisted training is significantly better than the baseline predictive models but exhibits only slight improvement over latent variables obtained with classical CD-learning. To demonstrate the training efficiency of the energy-based model with QC-assisted generative training, the training curves obtained over multiple runs are plotted in FIG. 5A. As evident from these, QC-assisted generative training progresses at a rate similar to that of CD-learning but converges to a significantly lower free energy difference value. Additionally, by drawing samples directly from the quantum annealer, QC-assisted learning requires O (|y|·|h|·(|y|+|h|)) less algebraic operations than one-step CD-learning for each step of the training process, where y and h represent the one-hot encoded vector denoting the property target label and the latent vector, respectively. FIG. 5B represents the optimization trajectory of searching for molecules within the chemical space defined by a fixed target property and given atomic identity. For comparison purposes, we attempt to solve the optimization problem with a constrained Bayesian optimization technique using the Gaussian process as the surrogate model [51]. Bayesian optimization is chosen for comparison as it not only allows to circumvent the complex functional form of a highly nonlinear optimization problem, but also utilizes a sequential procedure to improve the objective which is similar to that of the QC-based approximate optimization technique described herein. As compared to the constrained Bayesian optimization technique, the QC-assisted optimization technique of the present embodiments quickly explores better objective function values to yield local minima, as seen in FIG. 5B. FIG. 5C also presents a comparison of computational times required to solve the QUBO problems of varying sizes occurred during molecular generation for different atomic compositions and property targets with both simulated annealing implemented on a classical computer and quantum annealing. The presented comparisons clearly demonstrate that the QC-assisted techniques serve as an enhanced alternative to conventional learning and optimization methods implemented on classical computers.

Computational Issues: One of the limitations of QC-assisted strategy for molecular generation is that the exploration of molecular candidates exhibiting target properties can only be performed within certain ranges only. Although this constrained exploration of the chemical space provides better flexibility in identifying molecules with known atomic identities that satisfy the property requirements, determining molecules with exactly given properties may be difficult. In contrast to the trained deep generative models that can sample molecules for specific target properties, we apply an optimization procedure to perform a guided search for molecules that satisfy the target requirements. This is evident from the presented properties of generated molecules obtained with various molecular design techniques in FIG. 2A and Table S3. Within our computational experiments, we observed that the molecules that exhibit desired properties were found generally towards the end of the optimization procedure, which requires a higher computational effort than direct sampling with deep generative models. We acknowledge that the present embodiments exhibit certain limitations as compared to the generative deep learning systems for molecular generation in literature. Despite these limitations and the challenges associated with near-term QC, comparisons of property prediction with various baselines followed by an analysis of the generated molecules substantiates our objective of developing a QC-based molecular design framework capable of generating molecules with high efficacy. Areas for future improvements involve the development of generative models that could be trained efficiently with QC-based strategies and are capable of directly sampling molecular candidates without any additional steps. Furthermore, the use of the presented QC-based strategies to study and generate chemical reactions could also be explored.

### Techniques

Molecular Representation: The first step towards constructing quantitative structure-property relationships is to transform the chemical information of molecules into descriptors or fingerprints. The numerical encoding of the molecular structures is necessary for their feature representation as well as for similarity searches during virtual screening. MACCS and ECFP are examples of substructure-based and circular fingerprints, respectively [52]. Although such fingerprints have been previously used for predictive modeling, they are expert-based representations that are constructed with expert knowledge governed by a set of rules [53]. In addition, the non-invertibility of molecular fingerprints further complicates the search for molecules within the chemical space that produce desired representations obtained with predictive models. As a result, we use a graph convolutional neural network to generate neural fingerprints that serve as input to the energy-based model. Graph convolutional networks allow for learning task-specific molecular features to achieve a competitive predictive performance [48]. To generate neural fingerprints, a graph convolutional neural network is constructed that operates on molecules structured as graphs with atoms as nodes and bonds as edges. The constructed network uses a convolution operator described in Ref. [48] followed by a linear transformation to generate hidden feature vectors for individual atoms. This convolution operator has distinct weight matrices for each possible degree of vertices in the input graph. A softmax operation along the node features is then performed for each node to facilitate information flow between the atomic features of the molecule. Finally, a global pooling operation is applied to the updated hidden node features to generate a fixed-length vector as the network output. It should be noted that the graph convolutional network is initialized with large random weights without training. This graph convolutional network yields a direct nonlinear mapping G_{c} between the molecular graph and its neural fingerprint f=G_{c}(X,A), where X represents the feature matrix for nodes and A represents the adjacency matrix of the graph. As described in more detail below, the neural fingerprints are validated through similarity comparisons with ECFP and MACCS molecular representations.

Drawing Samples from Quantum Annealer: Quantum annealing refers to a search technique that can be implemented on an AQC platform. Compared to simulated annealing, quantum annealing demonstrates convergence to the ground state with a larger probability under similar conditions [54]. Quantum annealers are made available by D-Wave Systems with access to its users through the cloud. The D-Wave system can only solve quadratic unconstrained binary optimization (QUBO) problems. The QUBO graph is then mapped onto the quantum processing unit (QPU), with edges between nodes as internal couplers and qubits as the nodes. D-Wave offers QPUs with a topological structure of interconnected qubits forming either the Chimera graph or Pegasus topology [55]. To physically perform this mapping from variables to qubits, the process of minor-embedding is required [56,57]. Solving a QUBO problem on the D-Wave QPU comprises three main steps. First, the QUBO problem graph is embedded on the QPU. Second, low-energy solutions of the problem Hamiltonian are obtained through quantum annealing. Third, the solution is unembedded and is returned to the user. Notably, the process of finding an optimal graph-minor embedding is NP-hard. However, heuristic algorithms exist for finding graph-minors [58], and the D-Wave system implements its automatic embedding and unembedding tools [59]. Nevertheless, special care must be taken to formulate the smallest QUBOs possible to facilitate the embedding process for large-scale problems, to enable the solution on such quantum devices. Quantum annealers allow for implementation of a target Ising Hamiltonian with only pairwise interactions and local terms as

$${H}_{Ising} = {\sum }_{i , j} {J}_{ij} {σ}_{i}^{z} {σ}_{j}^{z} + {\sum }_{i} {h}_{i} {σ}_{i}^{z}$$

and can generate independent configurations from noisy Gibbs distributions [60]. All computational experiments involving the use of quantum annealers in this work are performed with the D-Wave Advantage quantum processor that offers at least 5,000 qubits and 35,000 couplers [61]. Each annealing run used for both generative training and optimization is performed for 20 μs on this quantum processor.

Energy-Based Model: We develop an energy-based model for molecular property prediction that can utilize QC techniques for efficient learning. Energy-based models can learn the distribution of data by associating an unnormalized probability value or energy to each data point. Additionally, the difficulty in sampling from such models allows us to explore alternatives for classical approximation techniques with quantum sampling facilitated by a quantum computer. In this work, we adopt a conditional generative model called conditional restricted Boltzmann machine (CRBM) to incorporate molecular property targets as binary variables. CRBM is a nonlinear generative model that can capture the conditional probability of observed data and has been previously applied for time series generation [62]. Although energy-based models are typically used for generative modeling, they can also be used for classification tasks [63]. Owing to their rich expressivity of latent variables and modeling flexibility, we use a conditional energy-based model for the supervised learning task of molecular property prediction.

An input to the energy-based model is a fixed-length vector description of the molecule under consideration captured by the neural fingerprint. In order to learn the conditional distribution p(y|f) of property targets y given the fingerprint f, we construct a CRBM network that defines a conditional joint distribution over f and latent variables h denoted by p_{θ}(y,h|f). The property targets are encoded into one-hot vectors through discrete binning of the target property values to minimize the effects of small observation errors. The hidden latent variables are modeled as binary units and can provide auxiliary information. The marginal conditional distribution of the target property y given the molecular fingerprint f can then be written as shown in Eq. (1), where F_{θ}(y, f) represents the associated free energy and Z(f) denotes the partition function, as defined in the Supplementary Information below. The constructed conditional energy-based model can be trained over a dataset D by maximizing the conditional log-likelihood of the input data as max_{θ}Σ_{d∈D} log p_{θ}(y^{d}|f^{d}). In order to apply gradient ascent for this maximization problem, the gradients of conditional log-likelihood can be analytically computed which leads to the parameter update rules as derived in the Supplementary Information. The [symbol] · [symbol] _{data} terms in the update rules can be easily computed with training data comprising of molecular fingerprints and properties for different molecules, however, computing the [symbol] · [symbol] ₚ_{_{θ}}_{(y, h|f)} exactly requires evaluating an expectation over p_{θ}(y,h|f). Estimation of this expectation value is computationally intractable due to the complexity stemming from the partition function Z(f) and requires appropriate approximation techniques in a practical setting.

$${p}_{θ} ( y \vert f ) = \frac{exp ( - {F}_{θ} ( y , f ) )}{Z ( f )} \qquad ( 1 )$$

Contrastive divergence (CD) learning can approximate the intractable expectations with Gibbs sampling [44] and has been extensively used for training energy-based models. CD-learning can, however, produce biased estimates of the conditional log-likelihood gradients and may even fail to converge in some cases. Although improved variants of CD learning can tackle such issues, the improvement in training performance is obtained at the expense of increased computational burden [63]. To overcome the limitations of classical approximation techniques, we incorporate QC-assisted training methodologies for efficient training of our energy-based model. p_{θ}(y,h|f) is a Boltzmann distribution also termed as Gibbs distribution expressed in the form of p(s)=exp(−E(s))/Z, wherein the energy E resembles an Ising Hamiltonian. As a result, we exploit quantum annealers to model p_{θ}(y,h|f) and draw samples from them to train the constructed energy-based model. The target Ising Hamiltonian on the quantum annealers is specified through the local fields h and pairwise couplings J. These input parameters are computed by mapping the energy function of the conditional energy-based model

$$E ( f , h ) = - {\sum }_{i} ( {b}_{i} + {\sum }_{k} {W}_{1}^{ki} {f}_{k} ) {y}_{i} - {\sum }_{j} ( {c}_{j} + {\sum }_{k} {W}_{2}^{kj} {f}_{k} ) {h}_{j} - {\sum }_{i , j} {W}^{ij} {y}_{i} {h}_{j}$$

to the Ising Hamiltonian. This energy function can be cast as a logical bipartite graph with y and h as its nodes and the parameters θ=(W₁, W₂, W, b, c) and fingerprint f governing its node and edge weights. In order to draw samples from the quantum annealer, we map the bipartite graph onto the physical hardware graph of qubits and couplers. It is important to note that the connectivity of the hardware graph poses certain restrictions on the size of logical graphs that can be embedded onto the QPU, so careful consideration of the degree of the logical bipartite graph dependent on the sizes of fingerprint and latent variable vectors is required. We obtain an embedding scheme for mapping the energy function onto the quantum annealer with a heuristic tool [58] which is used repeatedly while drawing samples from the Boltzmann distribution. For each update step of the model parameters, several samples are drawn from p_{θ}(y,h|f) by performing multiple annealing runs to estimate the expectations [symbol] y [symbol] _{P}_{_{θ}}_{(y,h|f)}, [symbol] h [symbol] ₚ_{_{θ}}_{(y,h|f)}, and [symbol] y^{T}h [symbol] _{P}_{_{θ}}_{(y,h|f)}.

Property Estimation: The trained conditional energy-based model yields the marginal distribution p_{θ}(y|f) due to the generative modeling approach. For property prediction tasks, we can estimate the target property by computing the latent representations with p_{θ}(h|y, f) and training a feedforward neural network using these representations as input. For molecules with unknown targets, the latent variables can be estimated by first determining the appropriate target label y with maximization of the conditional log-likelihood given a molecular fingerprint as max_{y} log p(y|f), which is followed by computing the latent representations and passing them through the feedforward network to predict exact property targets. Fixed length neural fingerprints of size 256 are generated with the fixed weights GraphConv network that serves as input to the conditional energy-based model in addition to the corresponding property labels encoded as discrete bins. We set the dimension of latent space representation to 64 during the construction of the energy-based model. This conditional energy-based model is trained with the molecules and their property targets in training set by drawing samples from a quantum annealer. The same energy-based model is also trained with CD-learning [44] carried out on a classical computer to discern any benefits of the QC-based approach over its classical counterpart. The training procedure of the conditional energy-based models is monitored with the difference between the free energy of original and reconstructed target labels. Furthermore, three baseline models are trained to predict molecular properties for benchmarking purposes. Two fully-connected networks that use ECFP and MACCS fingerprints [52] of lengths 2048 and 512 as input are trained using backpropagation with the Adam optimizer [64]. The same procedure is used to train a GraphConv model that operates directly on molecules cast as graph-structured data to predict their properties directly. The training for all models is terminated when the validation metrics fail to improve over consecutive steps, and their predictive performance is evaluated with mean absolute errors computed on the test set.

Optimization Strategy for Molecular Design: Generation of molecules by inverting the structure-property relationships obtained with the trained conditional energy-based model can be cast as an optimization problem. For a given target property range, the corresponding one-hot label can be determined as y, then the optimization problem deals with the objective min_{X,A}F_{θ}(y, f) and is subject to the constraint f=G_{c}(X, A) in addition to the structural constraints Ω that guarantee the generation of feasible molecules. This problem can be simplified by fixing the feature matrix of atoms X in the molecule provided either by guided intuition or reference molecules, which leads to a search of the chemical space defined by the given atomic identities. Despite this, the optimization problem min_{A}{F_{θ}(y, f)|f=G_{c}(X, A); A∈Ω} is a constrained nonlinear optimization problem with discrete-continuous variables and is challenging to solve with off-the-shelf solvers. To tackle this problem, we develop a QC-assisted approximate optimization technique that is capable of efficiently navigating the chemical space defined by the given atomic identities and the desired property targets.

In order to exploit QC for optimization, it is important to formulate problems that are compatible with the specific quantum hardware. It should be noted that both circuit model quantum devices and quantum annealers can natively solve QUBO problems. The main idea behind the solution technique is the use of a weighted linear model Σ_{i,j>i}βᵢⱼAᵢⱼ that serves as a surrogate model to approximate the objective function F_{θ}(y, G_{c}(X,A)). Since the adjacency matrix is symmetric, A≡{Aᵢⱼ∈{0,1}|j>i, ∀i} form the variables for the molecular generation problem. In addition, the set of structural constraints Ω comprises inequalities of the form Σ_{j>i}Aᵢⱼ+Σ_{i>j}Aⱼᵢ≤vᵢ where vᵢ represents the valency of constituent atom i. These structural constraints can be modeled as a QUBO problem denoted by Q_{c} such that arg min_{A}Q_{c}∈Ω. This QUBO problem can be formulated as Q_{c}=ΣᵢQᵢ where Qᵢ is given by:

$${Q}_{i} = ( 1 - {v}_{i} ) ( {\sum }_{j > i} {A}_{ij} + {\sum }_{i > j} {A}_{ji} ) + {\sum }_{j > i} {\sum }_{i > k} {A}_{ij} {A}_{ki} + 2 ( {\sum }_{j > i} {\sum }_{k > j} {A}_{ij} {A}_{ik} + {\sum }_{i > j} {\sum }_{j > k} {A}_{ji} {A}_{ki} ) . \qquad ( 2 )$$

Similar to Bayesian optimization [65], the QC-based approximate optimization technique of the present embodiments implements two phases. The primary phase involves random exploration of the chemical space to generate molecules and computing the associated objective function values. This exploration phase is performed by using a quantum annealer to solve min_{A}Q_{c} to generate feasible molecules. The collected samples and the objective function values are recorded as

$$D ≡ {( {A}^{i} , {F}_{θ}^{i} )}_{i = 1 , …N}$$

where N indicates the number of exploration steps. The secondary phase of the solution technique adopts a data-driven approach to train the surrogate model by minimizing the squared error as

$${min}_{β} {\sum }_{i ∈ D} {( {F}_{θ}^{i} - {β}^{T} A )}^{2}$$

to yield least square estimates for β. Sampling a molecule in this phase then involves solving the QUBO min_{A}Σ_{i,j>i}βᵢⱼ+λQ_{c} where λ is a fixed multiplier that satisfies λ>>∥β∥_{∞}. The obtained molecule and the observed objective value function are further appended to D. During each step of the second phase, the least square estimates are updated over the dataset D, followed by solving the updated QUBO problem. This ensures that the adjacency matrix is sampled such that it approximately minimizes the surrogate model while maintaining the structural constraints. It is important to note that the QUBO problems in both phases of the QC-based optimization technique can be cast as a fully connected graph. This graph can be mapped onto the physical hardware graph of the quantum annealer and is subjected to restrictions posed by the connectivity of the specific device. As the connectivity of the logical graph cast by the QUBO problems remains consistent throughout the optimization procedure, we obtain an embedding scheme for the mapping of the logical graph to physical graph using a heuristic tool [58]. For a given optimization problem, the embedding scheme is determined only once and is reused over all sampling steps of the QC-based approximate optimization technique.

Generating Molecules: To demonstrate the viability of the QC-assisted optimization technique for molecular generation, we conduct multiple computational experiments with various property targets. The conditional energy-based model trained with QC-assisted generative training is used as the structure-property relationship for inversion with the QC-based approximate optimization. For a given property target, appropriate target labels are obtained, and the corresponding conditional energy-based model is used to compute the objective function values for sampled molecules. Atomic identities of the molecules in the test set are used to search the associated chemical spaces with the solution technique for molecular candidates that satisfies target property requirements. Ten exploration steps are performed for each optimization problem, followed by 90 sampling steps to minimize the associated free energy of the trained conditional energy-based model. The molecules generated with the QC-assisted optimization technique are recorded for various conditions on properties like partition coefficient and drug-likeness. Additionally, we also calculate the QED, SAS, and Log P values for all generated molecules using the RDKit package [40] to validate the performance of the QC-based optimization technique.

### Supplemental Information

Data Processing: We first collected the SMILES identifiers of 12,000 molecules and identified their atomic and bond features with the RDKit package [40]. PyTorch Geometric (PyG) is a graph neural network package based on PyTorch and was used to construct molecular graphs for recording structural information and molecular properties. The molecular dataset is partitioned into a training set of 10,000 molecular graphs and property targets in addition to validation and test sets of size 1,000 each [39]. For the training, validation, and test datasets, a batched Dataset container in PyG was further constructed, which includes a batch of molecular graphs as one large disconnected graph. For each molecular property, target labels were also generated by constructing several bins that discretize the target space. To create a balanced dataset, we perform equal frequency binning on the samples in the training set to obtain the required set of bins. The generated bins for each property target along with their frequency, are reported in Table S1.

TABLE S1
Numeric ranges for different molecular properties
are obtained by performing equal frequency binning
and are used to generate target labels.

| QED | SAS | LogP |
|---|---|---|
| 0.138-0.526 | 1.399-2.122 | −4.580-0.522 |
| 0.526-0.615 | 2.122-2.316 | 0.522-1.295 |
| 0.615-0.673 | 2.316-2.506 | 1.295-1.799 |
| 0.673-0.721 | 2.506-2.694 | 1.799-2.222 |
| 0.721-0.759 | 2.694-2.889 | 2.222-2.583 |
| 0.759-0.790 | 2.889-3.093 | 2.583-2.945 |
| 0.790-0.819 | 3.093-3.365 | 2.945-3.313 |
| 0.819-0.847 | 3.365-3.754 | 3.313-3.688 |
| 0.847-0.881 | 3.754-4.232 | 3.688-4.166 |
| 0.881-0.947 | 4.232-6.791 | 4.166-6.663 |

Predictive Performance: Various metrics for the energy-based model trained with QC-assisted learning are presented in Table S2, wherein prediction errors for the SAS scores are skewed towards the higher range as compared to the other molecular properties. We also plot the principal component mappings of latent representations for 1,000 randomly selected molecules obtained with the energy-based model trained with quantum generative training in FIGS. 6A-6C. Distinctions for different property target values are apparent from the compressed representation along the principal components. Such discernible patterns in the latent representations with respect to the molecular properties indicate the construction of robust latent space that can be utilized for accurate property prediction tasks.

TABLE S2
Predictive performance of the energy-based model trained with
QC-assisted learning for molecular properties along with the
statistical information at the end of the training process.

| Molecular Property | RMSE | MARE |
|---|---|---|
| Quantitative drug likeness (QED) | 0.759 | 1.45 × 10⁻⁵ |
| Synthetic accessibility score (SAS) | 0.914 | 4.31 × 10⁻⁵ |
| Partition coefficient (LogP) | 0.823 | 9.12 × 10⁻⁶ |

Generation Efficacy: Table S3 represents the average and standard deviation values presented for each target condition are calculated over the observed properties of the generated molecules with the SSVAE [35] and CVAE [49] approaches to ensure the reliability of the molecular generation process subject to conditions. As evident from these values, the observed molecular properties lie beyond the required target range for both of the variational autoencoder-based approaches. Although lower accessibility scores present opportunities for easier synthesis, inability to satisfy target property requirements casts doubt on the constrained sampling capabilities of these baseline molecular design techniques.

TABLE S3
Molecule generation efficacy of the baseline molecular design techniques is described through
different properties computed for the generated molecules for specified target requirements.

| Target | SSVAE | | | CVAE | | |
|---|---|---|---|---|---|---|
| Condition | QED | SAS | LogP | QED | SAS | LogP |
| 0.527 ≤ QED < 0.615 | 0.78 ± 0.105 | 2.70 ± 0.882 | 2.94 ± 1.230 | 0.80 ± 0.119 | 2.55 ± 0.722 | 3.40 ± 0.947 |
| 0.674 ≤ QED < 0.721 | 0.81 ± 0.103 | 2.73 ± 0.803 | 2.71 ± 1.074 | 0.83 ± 0.063 | 2.56 ± 0.583 | 3.26 ± 1.00 |
| 0.721 ≤ QED < 0.76 | 0.79 ± 0.109 | 2.69 ± 0.871 | 2.88 ± 1.142 | 0.86 ± 0.055 | 2.62 ± 0.318 | 2.96 ± 0.675 |
| 0.76 ≤ QED < 0.79 | 0.81 ± 0.098 | 2.66 ± 0.713 | 2.83 ± 0.970 | 0.83 ± 0.113 | 2.22 ± 0.223 | 2.80 ± 0.699 |
| 0.82 ≤ QED < 0.847 | 0.81 ± 0.089 | 2.77 ± 0.789 | 2.71 ± 1.151 | 0.82 ± 0.080 | 2.66 ± 0.709 | 2.88 ± 1.056 |
| 0.522 ≤ LogP < 1.295 | 0.81 ± 0.071 | 1.51 ± 0.197 | 1.89 ± 0.663 | 0.78 ± 0.109 | 2.58 ± 0.42 | 1.62 ± 1.036 |
| 1.799 ≤ LogP < 2.223 | 0.84 ± 0.061 | 1.39 ± 0.177 | 2.64 ± 0.496 | 0.82 ± 0.059 | 2.25 ± 0.389 | 2.69 ± 0.860 |
| 2.223 ≤ LogP < 2.584 | 0.85 ± 0.057 | 1.33 ± 0.148 | 2.87 ± 0.397 | 0.81 ± 0.072 | 2.48 ± 0.333 | 3.29 ± 0.958 |
| 2.584 ≤ LogP < 2.946 | 0.86 ± 0.062 | 1.37 ± 0.215 | 3.00 ± 0.304 | 0.78 ± 0.073 | 2.94 ± 1.029 | 3.75 ± 1.339 |
| 3.314 ≤ LogP < 3.688 | 0.86 ± 0.055 | 1.31 ± 0.168 | 3.14 ± 0.282 | 0.79 ± 0.099 | 2.36 ± 0.431 | 3.89 ± 0.597 |

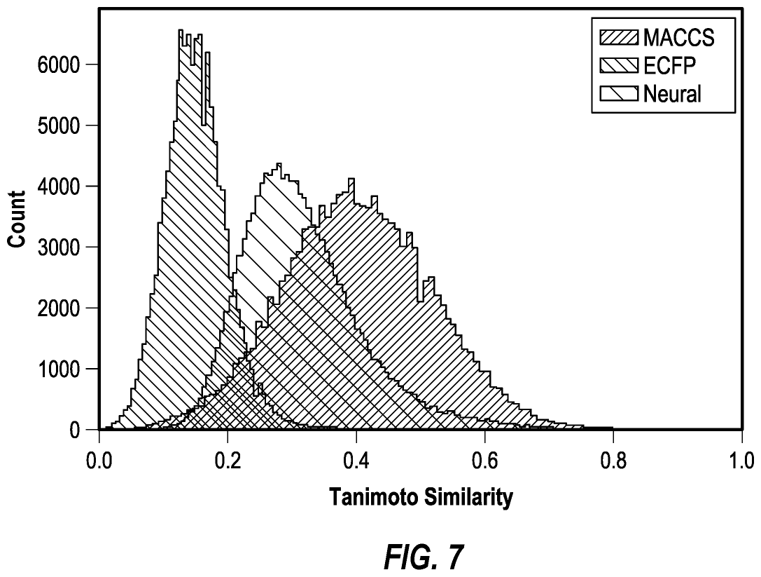

Molecular Representations: The GraphConv network is also constructed with PyG with the appropriate convolution operators. It should be noted that the neural fingerprints generated with GraphConv do not contain discrete values, as compared to the MACCS and ECFP fingerprints that provide binary representations. The molecular fingerprints allow us to measure the similarity between different molecules in a quantitative manner. Each element of binary fingerprints generally indicates the presence or absence of certain components like functional groups in a molecule. As this is not valid for continuous neural fingerprints generated with GraphConv, we can validate the similarities of different molecule pairs computed for each fingerprint. We randomly choose 500 query molecules among the training samples for this purpose and compute the Tanimoto similarities between each pair within this query set for each of the MACCS, ECFP, and neural fingerprints. The distribution of these similarities are plotted in FIG. 7. Compared to MACCS, the fingerprints obtained with GraphConv do not underestimate the similarity scores in the case of missing functional groups. Although the GraphConv network approximately mimics the rules used to compute the ECFP fingerprints [48], the neural fingerprints tend to generate smaller similarity scores than ECFP.

Constructing and Training the Energy-Based Model: The model parameters θ=(W₁, W₂, W, b, c) include weights W₁ and W₂ directed from conditional or fingerprint vector towards the visible and latent units, respectively, undirected weights W between the visible and latent variables, visible bias b, and hidden bias vector c. A joint probability distribution over target properties and latent variables conditioned on the molecular descriptors can be assigned with the energy functions as shown in Eq. SI. (3), wherein Z(f) is the normalization factor or the partition coefficient given by the expression Z(f)=Σ_{y}Σₕ exp(−E_{cond}(y,f)−E_{rbm}(y,h)). These energy functions capture the interactions between the fingerprints and both variables as well as between the target variables and the latent layer and are denoted by E_{cond}(y, f) and E_{rbm}(y,h), respectively in Eq. SI. (4)

$${p}_{θ} ( y , h \vert f ) = exp ( - {E}_{cond} ( y , f ) - {E}_{rbm} ( y , h ) ) / Z ( f ) \qquad SI . ( 3 )$$

$${E}_{cond} ( y , f ) = - {\sum }_{i , k} {W}_{1}^{ki} {y}_{i} {f}_{k} - {\sum }_{j , k} {W}_{2}^{kj} {h}_{j} {f}_{k} \qquad SI . ( 4 )$$

$${E}_{rbm} ( y , h ) = - {\sum }_{i} {b}_{i} {y}_{i} - {\sum }_{j} {c}_{j} {h}_{j} - {\sum }_{i , j} {W}_{t}^{ij} {y}_{i} {h}_{j}$$

$$p ( y \vert h , f ) = \frac{exp ( h \cdot {W}^{T} + f \cdot {W}_{1} + b )}{{\sum }_{y} exp ( h \cdot {W}^{T} + f \cdot {W}_{1} + b )} \qquad SI . ( 5 )$$

With the above information, the local conditional probabilities for the visible and hidden units can then be computed as p(h=1|f, y)=σ(f·W₂+y·W+c) and with Eq. SI. (5), where σ indicates the sigmoid function given by σ(x)=1/(1+exp(−x)). For a given molecular property, these probabilities can be used to sample latent variables and reconstruct input fingerprints. The marginal conditional distribution of the target property y given the molecular fingerprint f can then be written as shown in Eq. (1).

$${p}_{θ} ( y \vert f ) = \frac{{\sum }_{h} exp ( - {E}_{cond} ( y , f ) - {E}_{rbm} ( y , h ) )}{Z ( f )} = \frac{exp ( - {F}_{θ} ( y , f ) )}{Z ( f )} \qquad SI . ( 6 )$$

$${F}_{θ} ( y , f ) = - {\sum }_{i} {b}_{i} {y}_{i} - {\sum }_{i , k} {W}_{1}^{ki} {y}_{i} {f}_{k} - {\sum }_{j} log ( 1 + exp ( {c}_{j} + {\sum }_{i} {W}^{ij} {y}_{i} + {\sum }_{k} {W}_{2}^{kj} {f}_{k} ) ) \qquad SI . ( 7 )$$

$$Δ {W}_{1} ∝ f ( {〈 y 〉}_{data} - {〈 y 〉}_{{p}_{θ} ( y , h \vert f )} ) \qquad SI . ( 8 )$$

$$Δ {W}_{2} ∝ f ( {〈 h 〉}_{data} - {〈 h 〉}_{{p}_{θ} ( y , h \vert f )} ) \qquad SI . ( 9 )$$

$$Δ W ∝ {〈 {y}^{T} h 〉}_{data} - {〈 {y}^{T} h 〉}_{{p}_{θ} ( y , h \vert f )} \qquad SI . ( 10 )$$

$$Δ b ∝ {〈 y 〉}_{data} - {〈 y 〉}_{{p}_{θ} ( y , h \vert f )} \qquad SI . ( 11 )$$

$$Δ c ∝ {〈 h 〉}_{data} - {〈 h 〉}_{{p}_{θ} ( y , h \vert f )} \qquad SI . ( 12 )$$

The training rules SI. (8)-SI. (12) can be derived as follows by maximizing the log likelihood of marginal conditional distribution:

$$\frac{\partial }{\partial θ} log {p}_{θ} ( y \vert f ) = \frac{\partial }{\partial θ} log ( \frac{{\sum }_{g} exp ( - E ( y , h , f ) )}{Z ( f )} ) = \frac{\partial }{\partial θ} log ( {\sum }_{g} exp ( - E ( y , h , f ) ) ) - \frac{\partial }{\partial θ} log ( Z ( f ) ) = \frac{\frac{\partial }{\partial θ} {\sum }_{g} exp ( - E ( y , h , f ) )}{{\sum }_{g} exp ( - E ( y , h , f ) )} - \frac{\frac{\partial }{\partial θ} Z ( f )}{Z ( f )} = \frac{{\sum }_{g} exp ( - E ( y , h , f ) ) \cdot \frac{\partial }{\partial θ} ( - E ( y , h , f ) )}{{\sum }_{g} exp ( - E ( y , h , f ) )} - \frac{{\sum }_{y , h} exp ( - E ( y , h , f ) ) \cdot \frac{\partial }{\partial θ} ( - E ( y , h , f ) )}{Z ( f )} = {\sum }_{h} ( \frac{exp ( - E ( y , h , f ) )}{{\sum }_{{h}^{′}} exp ( - E ( y , {h}^{′} , f ) )} \cdot \frac{\partial }{\partial θ} ( - E ( y , h , f ) ) ) - {\sum }_{y , h} ( \frac{exp ( - E ( y , h , f ) )}{Z ( f )} \cdot \frac{\partial }{\partial θ} ( - E ( y , h , f ) ) ) = {\sum }_{h} p ( h \vert y , f ) \cdot \frac{\partial }{\partial θ} ( - E ( y , h , f ) ) - {\sum }_{y , h} p ( y , h \vert f ) \cdot \frac{\partial }{\partial θ} ( - E ( y , h , f ) ) = {\sum }_{h} p ( h \vert y , f ) \cdot \frac{\partial }{\partial θ} ( - E ( y , h , f ) ) - {\sum }_{y} p ( y \vert f ) {\sum }_{h} p ( h \vert y , f ) \cdot \frac{\partial }{\partial θ} ( - E ( y , h , f ) )$$

### Additional Embodiments

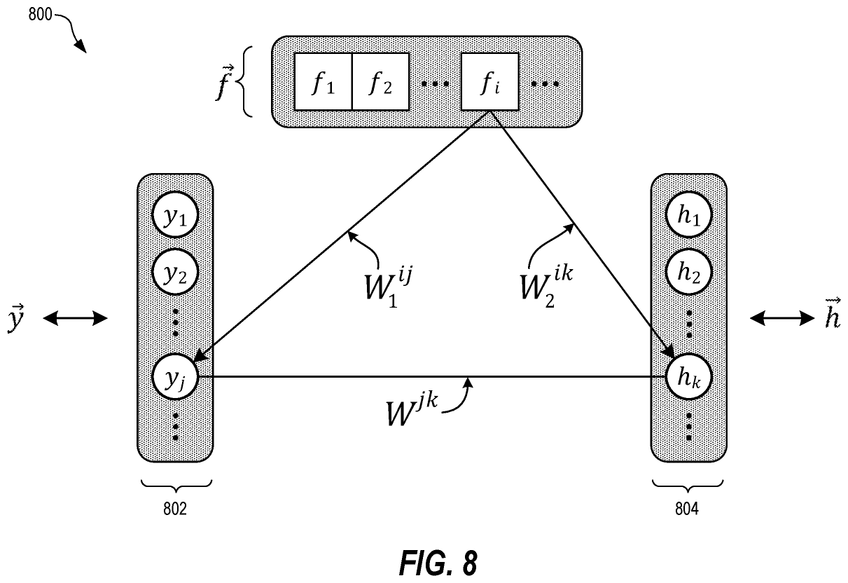

FIG. 8 illustrates a conditional restricted Boltzmann machine (CRBM) 800, which is one example of a machine-learning model that may be used with the present embodiments. The CRBM 800 is also one example of the conditional energy-based model shown in FIG. 1B. The CRBM 800 includes a plurality of visible nodes 802 and a plurality of hidden nodes 804. Each of the visible nodes 802 represents a visible binary variable yⱼ, where j is an index that runs from 1 to the number of the visible nodes 802. The ordered set of visible binary variables {y₁, . . . , yⱼ, . . . }≡y therefore forms a vector \vec{y} that is also referred to herein as a “target vector” or a “molecular property vector.” Since the vector \vec{y} and ordered set y are identical, the vector \vec{y} is sometimes indicated herein without an arrow (i.e., y).

Each of the hidden nodes 804 represents a hidden binary variable hₖ, where k is an index that runs from 1 to the number of the hidden nodes 804. The ordered set of hidden binary variables {h₁, . . . , hₖ, . . . }≡h therefore forms a vector \vec{h} that is also referred to herein as a “latent vector” or “hidden vector.” Since the vector \vec{h} and ordered set h are identical, the vector \vec{h} is sometimes indicated herein without an arrow (i.e., h). The vector \vec{h} exists in a latent vector space, or simply “latent space,” whose dimensionality (i.e., the number of hidden binary variables) is equal to the number of the hidden nodes 804.

Each of the visible nodes 802 directly connects to each of the hidden nodes 804 via a weight W^{jk}, where the superscripts indicate the row and column of a weight matrix W whose elements are the weights between the visible nodes 802 and the hidden nodes 804. The visible nodes 802 and hidden nodes 804 are fully connected in that each of the visible nodes 802 directly connects (i.e., has a single weight) to each of the hidden nodes 804, and vice versa. The visible nodes 802, hidden nodes 804, and weight matrix W collectively form a Boltzmann machine that is “restricted” in that none of the visible nodes 802 directly connect to each other and none of the hidden nodes 804 directly connect to each other.

The CRBM 800 is “conditional” in that it connects to a conditional vector \vec{f} having an ordered set of conditional-vector elements {f₁, . . . , fᵢ, . . . }≡{fᵢ}, where i is an index that runs from 1 to the number of these conditional-vector elements. Unlike the binary variables yⱼ and hₖ, the conditional-vector elements {fᵢ} may be continuous-valued or discrete-valued. Since the vector \vec{f} and ordered set f are identical, the vector \vec{f} is sometimes indicated herein without an arrow (i.e., f). The conditional vector \vec{f} is also referred to herein as a “molecular fingerprint,” “neural fingerprint,” or “feature vector.”

The conditional-vector element fᵢ directly connects to the visible binary variable vⱼ via a weight W₁^{ij}, where the superscripts indicate the row and column of a weight matrix W₁ whose elements are the weights between the conditional-vector elements {fᵢ} and the visible nodes 802. Similarly, the vector element fᵢ directly connects to the hidden binary variable hₖ via a weight W₂^{ik}, where the superscripts indicate the row and column of a weight matrix W₂ whose elements are the weights between the conditional-vector elements {fᵢ} and the hidden nodes 804.

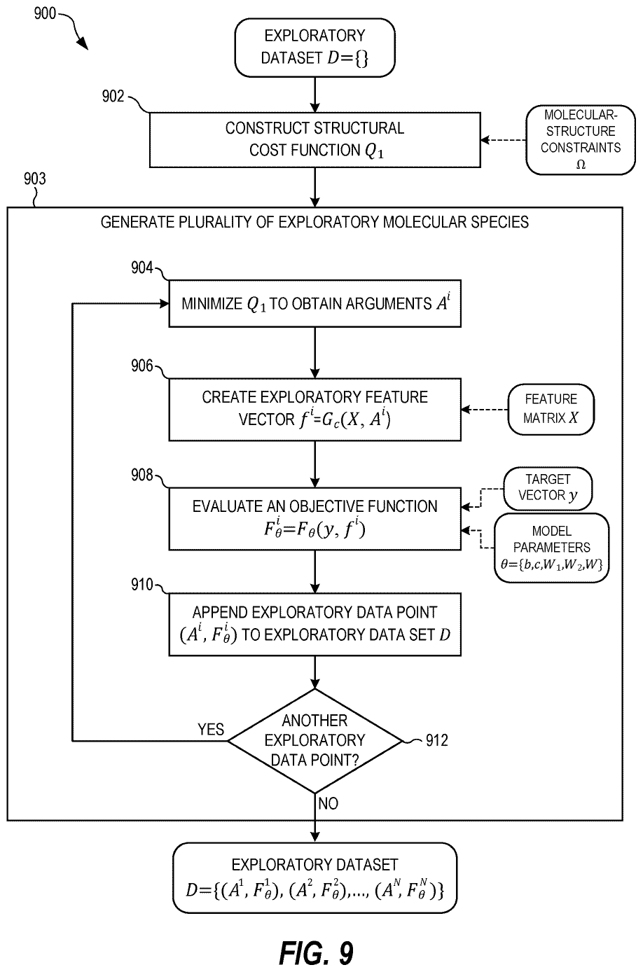

FIG. 9 is a flowchart of a method 900 for exploratory molecular design, in accordance with some of the present embodiments. As shown in FIG. 9, some embodiments of the method 900 start with an exploratory dataset D that is initially empty. In step 902 of the method 900, a structural cost function Q₁ is constructed. The structural cost function Q₁ has a plurality of first minima, each identifying, or corresponding to, a molecular species that satisfies one or more molecular-structure constraints Ω. As an example of the step 902, the function Q_{c} is constructed such that each of its minima corresponds to one of the structural constraints Ω, as described above in the section titled “Optimization Strategy for Molecular Design.” The molecular-structure constraints Ω may be in the form of inequalities involving the valency of each specified constituent atom.

In step 903 of the method 900, a plurality of exploratory molecular species are generated. The step 903 includes steps 904, 906, 908, 910, and 912. In the step 904, the structural cost function Q₁ is minimized to obtain a set of one or more arguments Aⁱ of a first minimum that is one of the plurality of first minima. Here, i is an index that numerically labels the plurality of exploratory molecular species that are generated. In some embodiments, each of the plurality of exploratory molecular species is formed from a plurality of specified constituent atoms. For example, the feature matrix X described above identifies an atomic species of each of the specified constituent atoms. In this case, the set of arguments Aⁱ of the first minimum may form an exploratory adjacency matrix whose elements identify atomic bonds between pairs of the specified constituent atoms. For clarity herein, this adjacency matrix is sometimes referred to with the symbol A.

In some embodiments, the step 904 is performed with a quantum computer. The quantum computer may be a quantum annealer that implements the minimization of the structural cost function Q₁ as a QUBO problem. However, another type of quantum computer may be used provided that the minimization can be implemented in a way that is compatible with the quantum computer's architecture (e.g., polynomial unconstrained binary optimization). For example, some minimization problems can be formulated for execution with a gate-model quantum computer or another type of quantum-computing architecture (e.g., topological quantum computing, cluster-state quantum computing, etc.). In other embodiments, the step 904 is performed solely via classical computing techniques, or a combination of classical and quantum computing techniques.

In step 906 of the method 900, an exploratory feature vector fⁱ is created based on the set of one or more arguments Aⁱ of the first minimum. The exploratory feature vector fⁱ identifies the corresponding exploratory molecular species within a feature vector space (or simply “feature space”). In some embodiments, the exploratory feature vector f is created by feeding the exploratory adjacency matrix and the plurality of specified constituent atoms into the trained GraphConv G_{c} (see the section above titled “Molecular Representation”). However, another type of trained machine-learning model may be used instead of, or in addition to, the trained GraphConv. Examples of such machine-learning models include, but are not limited to, recurrent neural networks and variational autoencoders.

In step 908 of the method 900, an objective function F_{θ} is evaluated with (i) the exploratory feature vector fⁱ, (ii) a target range of a molecular property, and (iii) model parameters θ of a trained machine-learning model to obtain an objective-function value F_{θ}ⁱ=F_{θ}(y, fⁱ) associated with the exploratory molecular species. In FIG. 9, θ=(b, c, W₁, W₂, W) is the set of model parameters of the CRBM 800 after the CRBM 800 has been trained. In addition, y is a one-hot target vector that encodes the target range of the molecular property. The objective-function value

$${F}_{θ}^{i}$$

is combined with the set of arguments Aⁱ of the first minimum to create an exploratory data point

$$( {A}^{i} , {F}_{θ}^{i} ) .$$

In the step 910 of the method 900, the exploratory data point

$$( {A}^{i} , {F}_{θ}^{i} )$$

is appended to the exploratory dataset D.

In one example of the step 908, the free energy of the CRBM 800 is used for the objective function F_{θ} (see the sections above titled “Optimization Strategy for Molecular Design” and “Constructing and Training the Energy-Based Model”). However, the free energy is only one type of the objective function F_{θ} that may be used with the method 900. The free energy is typically used when the trained machine-learning model is an energy-based model, such as the CRBM 800. However, other types of energy may be used for the objective function F_{θ}, depending on the type of energy-based model. Examples of other energies include, but are not limited to, Helmholtz energy and Gibbs energy. When the machine-learning model is not energy-based, the objective function F_{θ} may be, for example, an error-based function.

It is assumed in the step 908 that the trained machine-learning model was previously trained with a plurality of training samples. Each of these training samples includes a training feature vector that identifies a corresponding training molecular species within the feature space. The training sample also includes a training target vector that specifies a predefined range of values for each of one or more molecular properties. For each of these one or more molecular properties, the training molecular species has a value of the molecular property that lies within the respective predefined range of values.

The step 912 is a decision. If another exploratory data point is to be generated, the step 903 returns to the step 904. If no more exploratory data points are to be generated, the method 900 stops. The output of the method 900 is the exploratory dataset D populated with N exploratory data points

$$( {A}^{1} , {F}_{θ}^{1} ) , … , ( {A}^{N} , {F}_{θ}^{N} ) .$$

Iterations of the step 903 may continue for a fixed, predetermined value of the number N. In this case, the step 903 may be implemented, for example, with a simple for-loop. Alternatively, iterations may continue until some other condition is met. For example, the step 903 may continue iterating until the objective-function values of the exploratory dataset D obey or adhere to one or more statistic-based conditions.

In some embodiments, the method 900 further includes training an untrained machine-learning model with the plurality of training samples to obtain the trained machine-learning model. An example of such training is described above in the section titled “Constructing and Training the Energy-Based Model.” In some of these embodiments, training includes quantum sampling with a quantum computer to obtain samples and using the obtained samples to update one or more parameters of the untrained machine-learning model. The quantum sampling may be performed with a quantum annealer. The training may include generating the training target vector via one-hot encoding of the value of the molecular property. Alternatively, the training may be performed using only classical computing techniques, or a combination of classical and quantum computing techniques.

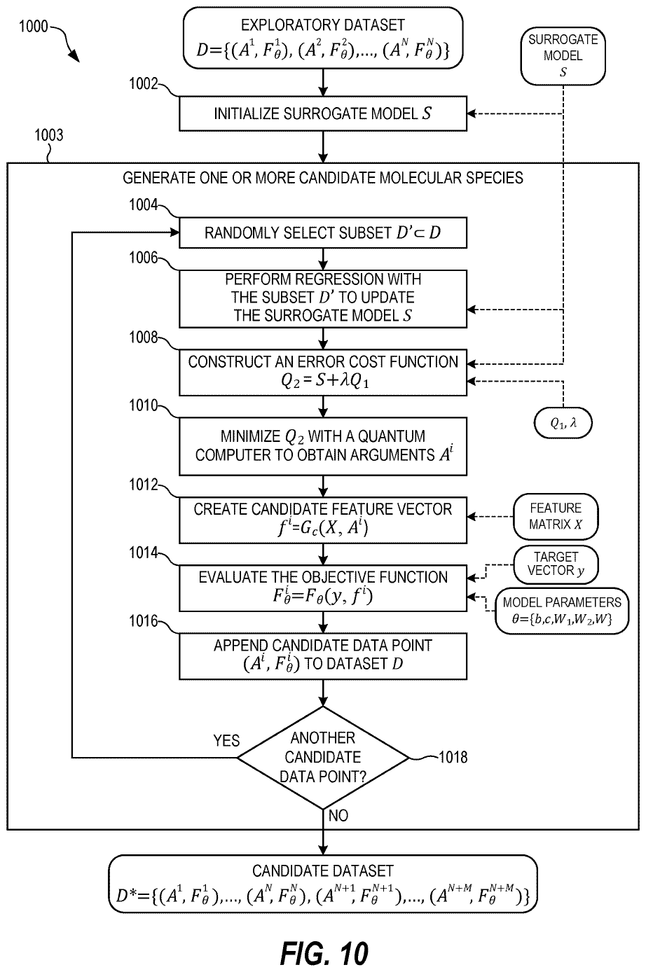

FIG. 10 is a flowchart of a method 1000 for generative molecular design, in accordance with some of the present embodiments. In the following description, it is assumed that the method 1000 is performed in combination with the method 900 of FIG. 9. However, the methods 900 and 1000 may be performed separately and independently from each other.

As shown in FIG. 10, some embodiments of the method 1000 start with the exploratory dataset D, as populated with the N exploratory data points

$$( {A}^{1} , {F}_{θ}^{1} ) … ( {A}^{N} , {F}_{θ}^{N} ) .$$

The method 1000 may also start with a surrogate model S. In some embodiments, the surrogate model S is a linear model, i.e., S=Σ_{i,j>i}βᵢⱼAᵢⱼ, where βᵢⱼ is an element of a matrix β of parameters of the linear model. However, the surrogate model S may alternatively be another type of parameterized model (e.g., polynomial, exponential, piece-wise, etc.). In embodiments where the surrogate model S is uninitialized (i.e., the parameters βᵢⱼ are undetermined or unspecified), the method 1000 may include the step 1002, in which the surrogate model S is initialized by selecting initial values for the parameters of the surrogate model S.

In step 1003 of the method 1000, one or more candidate molecular species are generated. The step 1003 includes steps 1004, 1006, 1008, and 1010. In some embodiments, the step 1003 further includes steps 1012, 1014, 1016, or 1018, or any combination thereof. In the step 1004, a subset D′ of exploratory data points are randomly selected from the exploratory dataset D. In the step 1006, regression is performed with the objective-function values of the subset D′ to update the surrogate model S such that the surrogate model S approximates the objective function F_{θ}. When the surrogate model S is a linear model, the regression may be linear regression (e.g., minimizing a sum of squared errors, as shown above in the section titled “Optimization Strategy for Molecular Design”). When the surrogate model S is a nonlinear model, the regression may be nonlinear regression (e.g., nonlinear least squares).

In the step 1008 of the method 1000, an error cost function Q₂ is constructed by combining the surrogate model S, as updated by the regression performed by the step 1006, with the structural cost function Q₁ such that each of a plurality of second minima of the error cost function Q₂ identifies a molecular species that minimizes the surrogate model S while satisfying the molecular-structure constraints Ω. In some embodiments, the error cost function Q₂ is a weighted sum of the structural cost function Q₁ and the surrogate model, e.g., Q₂=S+λQ₁, where λ is a fixed multiplier that is much greater than ∥β∥_{∞}. However, the error cost function Q₂ may have a different mathematical form without departing from the scope hereof.

In the step 1010 of the method 1000, the error cost function Q₂ is minimized with a quantum computer to obtain a set of one or more arguments Aⁱ of a second minimum that is one of the plurality of second minima. The quantum computer may be a quantum annealer that implements the minimization of the error cost function Q₂ as a QUBO problem. However, another type of quantum computer may be used provided that the minimization can be implemented in a way that is compatible with the quantum computer's architecture (e.g., polynomial unconstrained binary optimization). For example, some minimization problems can be formulated for execution with a gate-model quantum computer or another type of quantum-computing architecture. In other embodiments, the step 1004 is performed solely via classical computing techniques, or a combination of classical and quantum computing techniques.

The set of one or more arguments Aⁱ of the second minimum may be outputted to identify the molecular structure of the candidate molecular species that was generated. In some embodiments, each of the candidate molecular species is formed from a plurality of specified constituent atoms (e.g., the feature matrix X). In this case, the set of arguments Aⁱ of the second minimum may form a candidate adjacency matrix whose elements identify atomic bonds between pairs of the specified constituent atoms. The candidate adjacency matrix may then be outputted to identify, in part, the candidate molecular species that was generated. After outputting, the candidate adjacency matrix may be combined with the specified constituent atoms to reconstruct the molecular structure of the candidate molecular species.

In some embodiments, the method 1000 further includes the step 1012, in which a candidate feature vector fⁱ is created based on the set of one or more arguments Aⁱ of the second minimum. The candidate feature vector fⁱ identifies the corresponding candidate molecular species within the feature space. In some embodiments, and similar to the step 906 of FIG. 9, the candidate feature vector fⁱ is created by feeding the candidate adjacency matrix and specified constituent atoms into the trained GraphConv. However, another type of machine-learning model may be used instead of, or in addition to, the GraphConv to create the candidate feature vector fⁱ. The candidate feature vector fⁱ may then be outputted.

In some embodiments, the method 1000 further includes the step 1014, in which the objective function F_{θ} is evaluated with (i) the candidate feature vector fⁱ, (ii) the target range of a molecular property, and (iii) the model parameters θ of the trained machine-learning model to obtain an objective-function value

$${F}_{θ}^{i}$$

of the candidate molecular species.

In some embodiments, only one candidate molecular species is generated during the step 1003. In this case, the method 1000 may then output a first set of one or more arguments A¹ of the second minimum, a first candidate feature vector f¹, or a first objective-function value

$${F}_{θ}^{1} ,$$

or any combination thereof. In other embodiments, more than one candidate molecular species is generated during the step 1003. In this case, the step 1003 may implement a loop in which each iteration of the loop generates another one of the candidate molecular species.

In some of the embodiments in which the step 1003 implements a loop, the method 1000 further includes the step 1016 in which the set of one or more arguments Aⁱ is combined with the objection-function value

$${F}_{θ}^{i}$$

to create a candidate data point

$$( {A}^{i} , {F}_{θ}^{i} )$$

that is appended to the exploratory dataset D. The method 1000 may further include the step 1018, which is a decision to generate another candidate data point (i.e., another one of the candidate molecular species). If another candidate data point is to be generated, the step 1003 returns to the step 1004 to start another iteration. If no additional candidate data points are to be generated, then the method 1000 stops. The exploratory data set D is now a candidate data set D* with N+M data points

$$( {A}^{1} , {F}_{θ}^{1} ) , … , ( {A}^{N} , {F}_{θ}^{N} ) , ( {A}^{N + 1} , {F}_{θ}^{N + 1} ) , … ( {A}^{N + M} , {F}_{θ}^{N + M} ) ,$$

where the first N data points are the exploratory data points and the last M data points are the candidate data points. Since candidate data points are added to the exploratory data set D, one or more of these candidate data points may be selected as part of the subset D′.

The method 1000 may output any one or more pieces of information from the candidate data set D*. For example, the set of arguments Aⁱ having the smallest objective-function value

$${F}_{θ}^{i}$$

may be outputted as an optimum solution. The smallest objective-function value

$${F}_{θ}^{i}$$

may also be outputted to monitor convergence. The corresponding candidate feature vector fⁱ may also be outputted.

While the above discussion of the methods 900 and 1000 use the CRBM 800 of FIG. 8 as the trained machine-learning model, those trained in the art should recognize that the present embodiments may be adapted to work with other types of machine-learning models. For example, the trained machine-learning model may be a conditional non-energy-based model, an unconditional energy-based model, or a conditional energy-based model (e.g., the CRBM 800). Examples of unconditional and non-energy-based machine-learning models that will work with the present embodiments include unconditional variational autoencoders, conditional variational autoencoders, recurrent neural networks, feedforward neural networks, convolutional neural networks, and long/short-term neural networks. Some machine-learning models (e.g., any kind of Boltzmann machine and variational autoencoders) are best used for used for quantum annealing. Other types of machine-learning models are best for gate-model quantum computers.

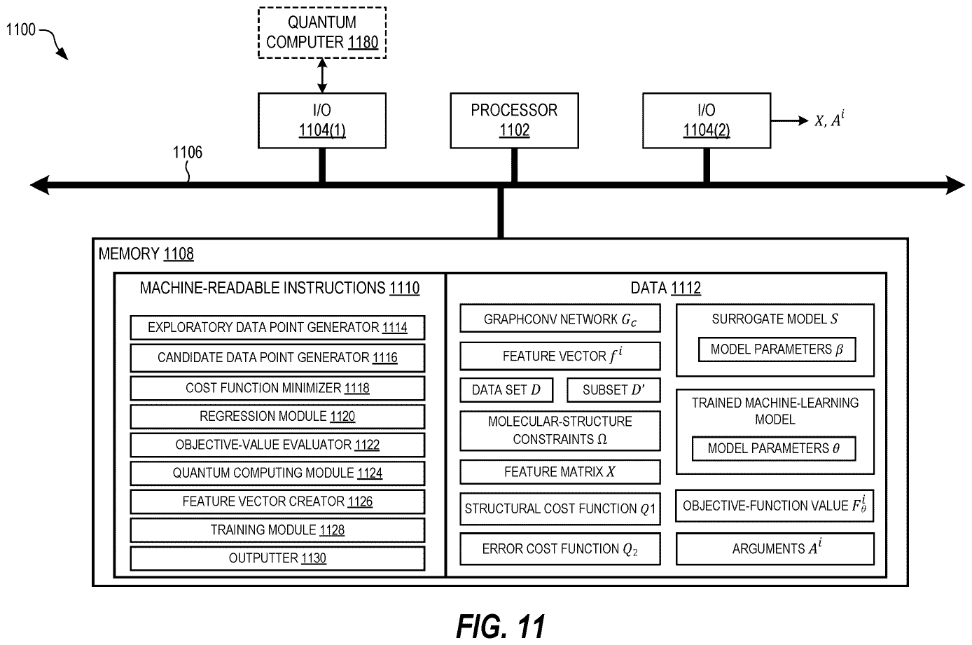

FIG. 11 is a functional diagram of a system 1100 for molecular design, in accordance with some of the present embodiments. The system 1100 includes a processor 1102 and a memory 1108 that communicate with each other over a system bus 1106. To communicate with external peripherals (e.g., monitors, keyboard, hard drives, memory cards, printers, etc.), computing devices (e.g., smart phones, tablets, etc.), and networks (e.g., Wi-Fi, Ethernet, Bluetooth, etc.), the system 1100 includes one or more input/output blocks 1104. In the example of FIG. 11, the system 1100 includes a first I/O block 1104(1) that interfaces with a quantum computer 1180. The system 1100 also includes a second I/O block 1104(2) that transmits results to another device. Although not shown in FIG. 11, the system 1100 may include more than two I/O blocks 1104 without departing from the scope hereof.

The system 1100 transmits instructions to the quantum computer 1180 via the I/O block 1104(1). These instructions control the quantum computer 1180 to perform minimization, sampling, or other quantum-computing techniques used by the present embodiments. The quantum computer 1180 transmits results (e.g., the arguments Aⁱ) back to the system 1100. The quantum computer 1180 may be a quantum annealer, a gate-model quantum computer, or another type of quantum computer known in the art. In some embodiments, the system 1100 includes the quantum computer 1180. In other embodiments, the system 1100 excludes the quantum computer 1180, in which case the quantum computer 1180 is maintained by, and accessed through, a third party.

The processor 1102 may be any type of circuit capable of performing logic, control, and input/output operations. For example, the processor 1102 may be a microprocessor having one or more central processing unit (CPU) cores, a graphics processing unit (GPU), a digital signal processor (DSP), a field-programmable gate array (FPGA), a system-on-chip (SoC), or a microcontroller unit (MCU), or any combination thereof. The processor 1102 may also include a memory controller, bus controller, and other components that manage data flow between the processor 1102, memory 1108, and I/O blocks 1104.

The memory 1108 stores machine-readable instructions 1110 that, when executed by the processor 1102, control the system 1100 to implement the functionality and methods described above. The machine-readable instructions 1110 include an exploratory data point generator 1114 that implements the step 903 of FIG. 9, a candidate data point generator 1116 that implements the step 1003 of FIG. 10, a cost function minimizer 1118 that implements the step 904 of FIG. 9 and the step 1010 of FIG. 10, a regression module 1120 that implements the step 1006 of FIG. 10, an objective-value evaluator 1122 that implements the step 908 of FIG. 9 and the step 1014 of FIG. 10, a quantum computing module 1124 that communicates with the quantum computer 1180 (e.g., as part of the cost function minimizer 1118), a feature vector creator 1126 that implements the step 906 of FIG. 9 and the step 1012 of FIG. 10, a training module 1128 that implements training of the machine-learning module, and an outputter 1130. The memory 1108 may store additional machine-readable instructions 1110 than shown in FIG. 11 without departing from the scope hereof.

The memory 1108 also stores data 1112 used by the processor 1102 when executing the machine-readable instructions 1110. This data 1112 includes the GraphConv network G_{c}, the feature vector fⁱ, the data set D, the subset D′, the molecular-structure constraints Ω, the feature matrix X, the structural cost function Q₁, the error cost function Q₂, the surrogate model S (include model parameters β), the trained machine-learning model (include model parameters θ), an objective-function value

$${F}_{θ}^{i} ,$$

and a set of arguments Aⁱ. The memory 1108 may store additional data 1112 than shown in FIG. 11 without departing from the scope hereof.

### REFERENCES

[1] Segal, D. Materials for the 21st Century. (Oxford University Press, 2017).
[2] Virshup, A. M., Contreras-Garcia, J., Wipf, P., Yang, W. & Beratan, D. N. Stochastic voyages into uncharted chemical space produce a representative library of all possible drug-like compounds. J Am Chem Soc 135, 7296-7303, doi: 10.1021/ja401184g (2013).
[3] Achenie, L., Venkatasubramanian, V. & Gani, R. Computer aided molecular design: theory and practice. (Elsevier, 2002).
[4] Feynman, R. P. Simulating physics with computers. International Journal of Theoretical Physics 21, 467-488, doi: 10.1007/BF02650179 (1982).
[5] Willett, P. Genetic algorithms in molecular recognition and design. Trends in Biotechnology 13, 516-521 (1995).
[6] Elton, D. C., Boukouvalas, Z., Fuge, M. D. & Chung, P. W. Deep learning for molecular design—a review of the state of the art. Molecular Systems Design & Engineering 4, 828-849 (2019).
[7] Austin, N. D., Sahinidis, N. V. & Trahan, D. W. Computer-aided molecular design: An introduction and review of tools, applications, and solution techniques. Chemical Engineering Research and Design 116, 2-26, doi: 10.1016/j.cherd.2016.10.014 (2016).
[8] Cao, Y., Romero, J. & Aspuru-Guzik, A. Potential of quantum computing for drug discovery. IBM Journal of Research and Development 62, 6:1-6:20, doi: 10.1147/JRD.2018.2888987 (2018).
[9] Ajagekar, A. & You, F. New frontiers of quantum computing in chemical engineering. Korean Journal of Chemical Engineering, doi: 10.1007/s11814-021-1027-6 (2022).
[10] Cao, Y. et al. Quantum Chemistry in the Age of Quantum Computing. Chemical Reviews 119, 10856-10915, doi: 10.1021/acs.chemrev.8b00803 (2019).
[11] Montanaro, A. Quantum algorithms: an overview. npj Quantum Information 2, 15023, doi: 10.1038/npjqi.2015.23 (2016).
[12] Biamonte, J. et al. Quantum machine learning. Nature 549, 195-202, doi: 10.1038/nature23474 (2017).
[13] Preskill, J. Quantum computing in the NISQ era and beyond. Quantum 2, 79 (2018).
[14] Robert, A., Barkoutsos, P. K., Woerner, S. & Tavernelli, I. Resource-efficient quantum algorithm for protein folding. npj Quantum Information 7, 38, doi: 10.1038/s41534-021-00368-4 (2021).
[15] Ajagekar, A., Humble, T. & You, F. Quantum computing based hybrid solution strategies for large-scale discrete-continuous optimization problems. Computers & Chemical Engineering 132, 106630, doi: 10.1016/j.compchemeng.2019.106630 (2020).
[16] Sliwoski, G., Kothiwale, S., Meiler, J. & Lowe, E. W. Computational methods in drug discovery. Pharmacological reviews 66, 334-395 (2014).
[17] Pyzer-Knapp, E. O., Suh, C., Gómez-Bombarelli, R., Aguilera-Iparraguirre, J. & Aspuru-Guzik, A. What is high-throughput virtual screening? A perspective from organic materials discovery. Annual Review of Materials Research 45, 195-216 (2015).
[18] Kim, S. et al. PubChem substance and compound databases. Nucleic acids research 44, D1202-D1213 (2016).
[19] Lehn, J. M. Dynamic combinatorial chemistry and virtual combinatorial libraries. Essays in Contemporary Chemistry: From Molecular Structure towards Biology, 307-326 (2001).
[20] Schneider, G. Trends in virtual combinatorial library design. Curr Med Chem 9, 2095-2101, doi: 10.2174/0929867023368755 (2002).
[21] Hautier, G., Jain, A. & Ong, S. P. From the computer to the laboratory: materials discovery and design using first-principles calculations. Journal of Materials Science 47, 7317-7340, doi: 10.1007/s10853-012-6424-0 (2012).
[22] Hansen, K. et al. Machine Learning Predictions of Molecular Properties: Accurate Many-Body Potentials and Nonlocality in Chemical Space. The Journal of Physical Chemistry Letters 6, 2326-2331, doi: 10.1021/acs.jpclett.5b00831 (2015).
[23] Montavon, G. et al. Machine learning of molecular electronic properties in chemical compound space. New Journal of Physics 15, 095003, doi: 10.1088/1367-2630/15/9/095003 (2013).
[24] Varnek, A. & Baskin, I. Machine Learning Methods for Property Prediction in Chemoinformatics: Quo Vadis? Journal of Chemical Information and Modeling 52, 1413-1437, doi: 10.1021/ci200409x (2012).
[25] Zunger, A. Inverse design in search of materials with target functionalities. Nature Reviews Chemistry 2, 0121, doi: 10.1038/s41570-018-0121 (2018).
[26] Sanchez-Lengeling, B. & Aspuru-Guzik, A. Inverse molecular design using machine learning: Generative models for matter engineering. Science 361, 360-365 (2018).
[27] Venkatasubramanian, V., Chan, K. & Caruthers, J. M. Computer-aided molecular design using genetic algorithms. Computers & Chemical Engineering 18, 833-844, doi: 10.1016/0098-1354 (93) E0023-3 (1994).
[28] Zhang, L., Cignitti, S. & Gani, R. Generic mathematical programming formulation and solution for computer-aided molecular design. Computers & Chemical Engineering 78, 79-84 (2015).
[29] Balamurugan, D., Yang, W. & Beratan, D. N. Exploring chemical space with discrete, gradient, and hybrid optimization methods. The Journal of chemical physics 129, 174105 (2008).
[30] Gupta, A. et al. Generative Recurrent Networks for De Novo Drug Design. Mol Inform 37, doi: 10.1002/minf.201700111 (2018).
[31] Segler, M. H. S., Kogej, T., Tyrchan, C. & Waller, M. P. Generating Focused Molecule Libraries for Drug Discovery with Recurrent Neural Networks. ACS Central Science 4, 120-131, doi: 10.1021/acscentsci.7b00512 (2018).
[32] Gómez-Bombarelli, R. et al. Automatic Chemical Design Using a Data-Driven Continuous Representation of Molecules. ACS Central Science 4, 268-276, doi: 10.1021/acscentsci.7b00572 (2018).
[33] Blaschke, T., Olivecrona, M., Engkvist, O., Bajorath, J. & Chen, H. Application of Generative Autoencoder in De Novo Molecular Design. Molecular informatics 37, 1700123, doi: 10.1002/minf.201700123 (2018).
[34] Sun, M. et al. Graph convolutional networks for computational drug development and discovery. Briefings in Bioinformatics 21, 919-935, doi: 10.1093/bib/bbz042 (2020).
[35] Kang, S. & Cho, K. Conditional Molecular Design with Deep Generative Models. Journal of Chemical Information and Modeling 59, 43-52, doi: 10.1021/acs.jcim.8b00263 (2019).
[36] You, J., Liu, B., Ying, Z., Pande, V. & Leskovec, J. Graph convolutional policy network for goal-directed molecular graph generation. Advances in neural information processing systems 31 (2018).
[37] Khatami, M. H., Mendes, U. C., Wiebe, N. & Kim, P. M. Gate-based Quantum Computing for Protein Design. arXiv preprint arXiv:2201.12459 (2022).
[38] Irwin, J. J. & Shoichet, B. K. ZINC—a free database of commercially available compounds for virtual screening. Journal of chemical information and modeling 45, 177-182, doi: 10.1021/ci049714+ (2005).
[39] Dwivedi, V. P., Joshi, C. K., Laurent, T., Bengio, Y. & Bresson, X. Benchmarking graph neural networks. arXiv preprint arXiv: 2003.00982 (2020).
[40] Landrum, G. RDKit: Open-source cheminformatics. (2010).<http://www.rdkit.org>.
[41] Bickerton, G. R., Paolini, G. V., Besnard, J., Muresan, S. & Hopkins, A. L. Quantifying the chemical beauty of drugs. Nature Chemistry 4, 90-98, doi: 10.1038/nchem.1243 (2012).
[42] Wildman, S. A. & Crippen, G. M. Prediction of Physicochemical Parameters by Atomic Contributions. Journal of Chemical Information and Computer Sciences 39, 868-873, doi: 10.1021/ci9903071 (1999).
[43] Ertl, P. & Schuffenhauer, A. Estimation of synthetic accessibility score of drug-like molecules based on molecular complexity and fragment contributions. Journal of Cheminformatics 1, 8, doi: 10.1186/1758-2946-1-8 (2009).
[44] Hinton, G. E. Training products of experts by minimizing contrastive divergence. Neural computation 14, 1771-1800 (2002).
[45] Carreira-Perpinan, M. A. & Hinton, G. in International workshop on artificial intelligence and statistics. 33-40 (PMLR).
[46] Rogers, D. & Hahn, M. Extended-connectivity fingerprints. Journal of chemical information and modeling 50, 742-754 (2010).
[47] Durant, J. L., Leland, B. A., Henry, D. R. & Nourse, J. G. Reoptimization of MDL keys for use in drug discovery. Journal of chemical information and computer sciences 42, 1273-1280 (2002).
[48] Duvenaud, D. K. et al. Convolutional networks on graphs for learning molecular fingerprints. Advances in neural information processing systems 28 (2015).
[49] Lim, J., Ryu, S., Kim, J. W. & Kim, W. Y. Molecular generative model based on conditional variational autoencoder for de novo molecular design. Journal of cheminformatics 10, 1-9 (2018).
[50] Weininger, D. SMILES, a chemical language and information system. 1. Introduction to methodology and encoding rules. Journal of chemical information and computer sciences 28, 31-36 (1988).
[51] Hernández-Lobato, J. M., Gelbart, M. A., Adams, R. P., Hoffman, M. W. & Ghahramani, Z. A general framework for constrained Bayesian optimization using information-based search. (2016).
[52] Cereto-Massagué, A. et al. Molecular fingerprint similarity search in virtual screening. Methods 71, 58-63, doi: 10.1016/j.ymeth.2014.08.005 (2015).
[53] Stepišnik, T., Škrlj, B., Wicker, J. & Kocev, D. A comprehensive comparison of molecular feature representations for use in predictive modeling. Computers in Biology and Medicine 130, 104197, doi: 10.1016/j.compbiomed.2020.104197 (2021).
[54] Kadowaki, T. & Nishimori, H. Quantum annealing in the transverse Ising model. Physical Review E—Statistical Physics, Plasmas, Fluids, and Related Interdisciplinary Topics 58, 5355-5363, doi: 10.1103/PhysRevE.58.5355 (1998).
[55] Dattani, N., Szalay, S. & Chancellor, N. Pegasus: The second connectivity graph for large-scale quantum annealing hardware. arXiv preprint arXiv:.07636 (2019).
[56] Klymko, C., Sullivan, B. D. & Humble, T. S. Adiabatic quantum programming: minor embedding with hard faults. Quantum Information Processing 13, 709-729, doi: 10.1007/s11128-013-0683-9 (2014).
[57] Okada, S., Ohzeki, M., Terabe, M. & Taguchi, S. Improving solutions by embedding larger subproblems in a D-Wave quantum annealer. Scientific Reports 9. (2019).
[58] Cai, J., Macready, W. G. & Roy, A. A practical heuristic for finding graph minors. arXiv preprint arXiv: 1406.2741 (2014).
[59] D-Wave. Obsolv documentation, <https://docs.ocean.dwavesys.com/projects/qbsolv> (2019).
[60] Vuffray, M., Coffrin, C., Kharkov, Y. A. & Lokhov, A. Y. Programmable quantum annealers as noisy gibbs samplers. arXiv preprint arXiv: 2012.08827 (2020).
[61] McGeoch, C., Farre, P., & Bernoudy, W. D-Wave Hybrid Solver Service+Advantage: Technology Update, dwavesys.com/resources (2020).
[62] Taylor, G. W., Hinton, G. E. & Roweis, S. Modeling human motion using binary latent variables. Advances in neural information processing systems 19 (2006).
[63] Mnih, V., Larochelle, H. & Hinton, G. E. Conditional restricted boltzmann machines for structured output prediction. arXiv preprint arXiv: 1202.3748 (2012).
[64] Kingma, D. P. & Ba, J. Adam: A method for stochastic optimization. arXiv preprint arXiv: 1412.6980 (2014).
[65] Frazier, P. I. A tutorial on Bayesian optimization. arXiv preprint arXiv: 1807.02811 (2018).

### Combinations of Features

Features described above as well as those claimed below may be combined in various ways without departing from the scope hereof. The following examples illustrate possible, non-limiting combinations of features and embodiments described above. It should be clear that other changes and modifications may be made to the present embodiments without departing from the spirit and scope of this invention:
(A1) A method for molecular design includes constructing a structural cost function having a plurality of first minima, each of the plurality of first minima identifying a molecular species that satisfies one or more molecular-structure constraints. The method also includes generating each of a plurality of exploratory molecular species by minimizing the structural cost function to obtain one or more arguments of one of the plurality of first minima; creating, based on the one or more arguments of said one of the plurality of first minima, an exploratory feature vector that identifies said each of the plurality of exploratory molecular species within a feature space; and evaluating an objective function with (i) the exploratory feature vector, (ii) a target range of a molecular property, and (iii) model parameters of a trained machine-learning model to obtain a respective one of a plurality of objective-function values. The method also includes generating a candidate molecular species by performing regression with a randomly selected subset of the plurality of objective-function values to update a surrogate model that approximates the objective function; constructing an error cost function by combining the surrogate model, as updated by the regression, and the structural cost function such that each of a plurality of second minima of the error cost function identifies a molecular species that minimizes the surrogate model while satisfying the one or more molecular-structure constraints; and minimizing, with a quantum computer, the error cost function to obtain one or more arguments of one of the plurality of second minima. The method also includes outputting the one or more arguments of said one of the plurality of second minima. The trained machine-learning model was trained with a plurality of training samples, each training sample of the plurality of training samples comprising (i) a training feature vector that identifies a training molecular species of said each training sample within the feature space and (ii) a training target vector that specifies a predefined range of values of the molecular property, the training molecular species having a value of the molecular property lying within the predefined range.
(A2) In the method denoted (A1), the method further includes creating, based on the one or more arguments of said one of the plurality of second minima, a candidate feature vector that identifies the candidate molecular species within the feature space.
(A3) In the method denoted (A2), the method further includes evaluating the objective function with (i) the candidate feature vector, (ii) the target range of a molecular property, and (iii) the model parameters of the trained machine-learning model to obtain an objective-function value of the candidate molecular species.
(A4) In any of the methods denoted (A1) to (A3), said minimizing the error cost function with a quantum computer includes minimizing the error cost function using a quantum annealer or a gate-model quantum computer.
(A5) In any of the methods denoted (A1) to (A4), the trained machine-learning model comprising a constrained machine-learning model.
(A6) In any of the methods denoted (A1) to (A4), the trained machine-learning model comprising an energy-based machine-learning model.
(A7) In the method denoted (A6), the energy-based machine-learning model includes a restricted Boltzmann machine.
(A8) In the method denoted (A7), the restricted Boltzmann machine includes a conditional restricted Boltzmann machine.
(A9) In any of the methods denoted (A1) to (A8), the method further includes training an untrained machine-learning model with the plurality of training samples to obtain the trained machine-learning model.
(A10) In the method denoted (A9), said training includes performing quantum sampling with a quantum computer to obtain samples. Said training also includes using the obtained samples to update one or more model parameters of the untrained machine-learning model.
(A11) In the method denoted (A10), said performing quantum sampling is performed with a quantum annealer.
(A12) In any of the methods denoted (A9) to (A11), said training includes generating the training target vector via one-hot encoding of the value of the molecular property.
(A13) In any of the methods denoted (A9) to (A12), for each of the plurality of training samples: the training molecular species is formed from a plurality of specified constituent atoms, a training adjacency matrix has elements that identify atomic bonds between pairs of the plurality of specified constituent atoms, and said training includes feeding the training adjacency matrix and identities of the plurality of specified constituent atoms into a graph convolutional neural network to generate the training feature vector.
(A14) In any of the methods denoted (A1) to (A13), said minimizing the structural cost function is performed, at least in part, by a quantum computer.
(A15) In the method denoted (A14), the quantum computer includes a quantum annealer.
(A16) In any of the methods denoted (A1) to (A15), said minimizing the error cost function includes performing quadratic unconstrained binary optimization.
(A17) In any of the methods denoted (A1) to (A16), said minimizing the structural cost function includes performing quadratic unconstrained binary optimization.
(A18) In any of the methods denoted (A1) to (A17), the candidate molecular species is formed from a plurality of specified constituent atoms. Furthermore, the one or more arguments of said one of the plurality of second minima includes a candidate adjacency matrix whose elements identify atomic bonds between pairs of the plurality of specified constituent atoms.
(A19) In the method denoted (A18), the method further includes creating a candidate feature vector by feeding the candidate adjacency matrix and identifications of the plurality of specified constituent atoms into a trained graph-convolutional neural network.
(A20) In any of the methods denoted (A1) to (A19), each of the plurality of exploratory molecular species is formed from a plurality of specified constituent atoms, the one or more arguments of said one of the plurality of structural minima include an exploratory adjacency matrix whose elements identify atomic bonds between pairs of the plurality of specified constituent atoms, and said creating the exploratory feature vector includes feeding the exploratory adjacency matrix and identifications of the plurality of specified constituent atoms into a trained graph-convolutional neural network.
(A21) In any of the methods denoted (A1) to (A20), the surrogate model is a linear regression model.
(A22) In any of the methods denoted (A1) to (A21), said generating the candidate molecular species includes generating a plurality of candidate molecular species by repeating said performing regression, said constructing the second objective function, and said minimizing the second objective function. Furthermore, said outputting includes outputting the one or more arguments of one or more of the plurality of candidate molecular species.
(A23) In the method denoted (A22), said outputting includes outputting the one or more arguments of at least a first candidate molecular species and a second candidate molecular species. The method further includes synthesizing the first candidate molecular species, synthesizing the second candidate molecular species, measuring a molecular property of the first candidate molecular species to obtain a first measured property value, measuring the molecular property of the second candidate molecular species to obtain a second measured property value, and comparing the first measured property value and the second measured property value.
(A24) In the method denoted (A23), the method further includes selecting either the first candidate molecular species or the second candidate molecule species based on said comparing.
(B1) A system for molecular design includes a processor and a memory in communication with the processor. The memory stores machine-readable instructions that, when executed by the processor, control the system to perform any of the methods denoted (A1) to (A24).
(B2) In the system denoted (B1), the system further includes the quantum computer.
(C1) A system for molecular design includes a processor and a memory in communication with the processor. The memory stores a trained machine-learning model that was trained with a plurality of training samples, each training sample of the plurality of training samples includes (i) a training feature vector that identifies a training molecular species of said each training sample within a feature space and (ii) a training target vector that specifies a predefined range of values of the molecular property, the training molecular species having a value of the molecular property lying within the predefined range. The memory stores machine-readable instructions that, when executed by the processor, control the system to construct a structural cost function having a plurality of first minima, each of the plurality of first minima identifying a molecular species that satisfies one or more molecular-structure constraints. The machine-readable instructions, when executed by the processor, also control the system to generate each of a plurality of exploratory molecular species by minimizing the structural cost function to obtain one or more arguments of one of the plurality of first minima; creating, based on the one or more arguments of said one of the plurality of first minima, an exploratory feature vector that identifies said each of the plurality of exploratory molecular species within the feature space; and evaluating an objective function with the exploratory feature vector, a target range of a molecular property, and model parameters of the trained machine-learning model to obtain a respective one of a plurality of objective-function values. The machine-readable instructions, when executed by the processor, also control the system to generate a candidate molecular species by performing regression with a randomly selected subset of the plurality of objective-function values to update a surrogate model that approximates the objective function; constructing an error cost function by combining the surrogate model, as updated by the regression, and the structural cost function such that each of a plurality of second minima of the error cost function identifies a molecular species that minimizes the surrogate model while satisfying the one or more molecular-structure constraints; and minimize, with a quantum computer, the error cost function to obtain one or more arguments of one of the plurality of second minima. The machine-readable instructions, when executed by the processor, also control the system to output the one or more arguments of said one of the plurality of second minima.
(C2) In the system denoted (C1), the system further includes the quantum computer.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

1. A method for molecular design, comprising:
constructing a structural cost function having a plurality of first minima, each of the plurality of first minima identifying a molecular species that satisfies one or more molecular-structure constraints;
generating each of a plurality of exploratory molecular species by:
minimizing the structural cost function to obtain one or more arguments of one of the plurality of first minima;
creating, based on the one or more arguments of said one of the plurality of first minima, an exploratory feature vector that identifies said each of the plurality of exploratory molecular species within a feature space; and
evaluating an objective function with (i) the exploratory feature vector, (ii) a target range of a molecular property, and (iii) model parameters of a trained machine-learning model to obtain a respective one of a plurality of objective-function values;
generating a candidate molecular species by:
performing regression with a randomly selected subset of the plurality of objective-function values to update a surrogate model that approximates the objective function;
constructing an error cost function by combining the surrogate model, as updated by the regression, and the structural cost function such that each of a plurality of second minima of the error cost function identifies a molecular species that minimizes the surrogate model while satisfying the one or more molecular-structure constraints; and
minimizing, with a quantum computer, the error cost function to obtain one or more arguments of one of the plurality of second minima; and
outputting the one or more arguments of said one of the plurality of second minima;
wherein the trained machine-learning model was trained with a plurality of training samples, each training sample of the plurality of training samples comprising (i) a training feature vector that identifies a training molecular species of said each training sample within the feature space and (ii) a training target vector that specifies a predefined range of values of the molecular property, the training molecular species having a value of the molecular property lying within the predefined range.

2. The method of claim 1, further comprising creating, based on the one or more arguments of said one of the plurality of second minima, a candidate feature vector that identifies the candidate molecular species within the feature space.

3. The method of claim 2, further comprising evaluating the objective function with (i) the candidate feature vector, (ii) the target range of a molecular property, and (iii) the model parameters of the trained machine-learning model to obtain an objective-function value of the candidate molecular species.

4. The method of claim 1, wherein said minimizing the error cost function with a quantum computer comprises minimizing the error cost function using a quantum annealer or a gate-model quantum computer.

5. The method of claim 1, the trained machine-learning model comprising a constrained machine-learning model.

6. The method of claim 1, the trained machine-learning model comprising an energy-based machine-learning model.

7. The method of claim 6, the energy-based machine-learning model comprising a restricted Boltzmann machine.

8. The method of claim 7, the restricted Boltzmann machine comprising a conditional restricted Boltzmann machine.

9. The method of claim 1, further comprising training an untrained machine-learning model with the plurality of training samples to obtain the trained machine-learning model.

10. The method of claim 9, wherein said training comprises:
performing quantum sampling with a quantum computer to obtain samples; and
using the obtained samples to update one or more model parameters of the untrained machine-learning model.

11. The method of claim 10, wherein said performing quantum sampling is performed with a quantum annealer.

12. The method of claim 9, wherein said training comprises generating the training target vector via one-hot encoding of the value of the molecular property.

13. The method of claim 9, wherein, for each of the plurality of training samples:
the training molecular species is formed from a plurality of specified constituent atoms;
a training adjacency matrix has elements that identify atomic bonds between pairs of the plurality of specified constituent atoms; and
said training comprises feeding the training adjacency matrix and identities of the plurality of specified constituent atoms into a graph convolutional neural network to generate the training feature vector.

14. The method of claim 1, wherein said minimizing the structural cost function is performed, at least in part, by a quantum computer.

15. The method of claim 14, the quantum computer comprising a quantum annealer.

16. The method of claim 1, wherein said minimizing the error cost function comprises performing quadratic unconstrained binary optimization.

17. The method of claim 1, wherein said minimizing the structural cost function comprises performing quadratic unconstrained binary optimization.

18. The method of claim 1, wherein:
the candidate molecular species is formed from a plurality of specified constituent atoms; and
the one or more arguments of said one of the plurality of second minima comprise a candidate adjacency matrix whose elements identify atomic bonds between pairs of the plurality of specified constituent atoms.

19. The method of claim 18, further comprising creating a candidate feature vector by feeding the candidate adjacency matrix and identifications of the plurality of specified constituent atoms into a trained graph-convolutional neural network.

20. The method of claim 1, wherein:
each of the plurality of exploratory molecular species is formed from a plurality of specified constituent atoms;
the one or more arguments of said one of the plurality of structural minima comprise an exploratory adjacency matrix whose elements identify atomic bonds between pairs of the plurality of specified constituent atoms; and
said creating the exploratory feature vector comprises feeding the exploratory adjacency matrix and identifications of the plurality of specified constituent atoms into a trained graph-convolutional neural network.

21. The method of claim 1, the surrogate model being a linear regression model.

22. The method of claim 1, wherein:
said generating the candidate molecular species comprises generating a plurality of candidate molecular species by repeating said performing regression, said constructing the second objective function, and said minimizing the second objective function; and
said outputting comprises outputting the one or more arguments of one or more of the plurality of candidate molecular species.

23. The method of claim 22, wherein:
said outputting comprises outputting the one or more arguments of at least a first candidate molecular species and a second candidate molecular species; and
the method further comprises:
synthesizing the first candidate molecular species;
synthesizing the second candidate molecular species;
measuring a molecular property of the first candidate molecular species to obtain a first measured property value;
measuring the molecular property of the second candidate molecular species to obtain a second measured property value; and
comparing the first measured property value and the second measured property value.

24. The method of claim 23, further comprising selecting either the first candidate molecular species or the second candidate molecular species based on said comparing.

25-26. (canceled)

27. A system for molecular design, comprising:
a processor; and
a memory in communication with the processor, the memory storing a trained machine-learning model that was trained with a plurality of training samples, each training sample of the plurality of training samples comprising (i) a training feature vector that identifies a training molecular species of said each training sample within a feature space and (ii) a training target vector that specifies a predefined range of values of a molecular property, the training molecular species having a value of the molecular property lying within the predefined range;
the memory storing machine-readable instructions that, when executed by the processor, control the system to:
construct a structural cost function having a plurality of first minima, each of the plurality of first minima identifying a molecular species that satisfies one or more molecular-structure constraints,
generate each of a plurality of exploratory molecular species by:
(i) minimizing the structural cost function to obtain one or more arguments of one of the plurality of first minima,
(ii) creating, based on the one or more arguments of said one of the plurality of first minima, an exploratory feature vector that identifies said each of the plurality of exploratory molecular species within the feature space, and
(iii) evaluating an objective function with the exploratory feature vector, a target range of the molecular property, and model parameters of the trained machine-learning model to obtain a respective one of a plurality of objective-function values,
generate a candidate molecular species by:
(i) performing regression with a randomly selected subset of the plurality of objective-function values to update a surrogate model that approximates the objective function,
(ii) constructing an error cost function by combining the surrogate model, as updated by the regression, and the structural cost function such that each of a plurality of second minima of the error cost function identifies a molecular species that minimizes the surrogate model while satisfying the one or more molecular-structure constraints, and
(iii) minimizing, with a quantum computer, the error cost function to obtain one or more arguments of one of the plurality of second minima, and
output the one or more arguments of said one of the plurality of second minima.

28. The system of claim 27, further comprising the quantum computer.
